SEQUENCE LISTING (29 sequences)

SEQ ID NO: 1 - DNA, 243 bp, Homo sapiens
atgtcgcagaacggagcgcccgggatgcaggaggagagcctgcagggctcctgggtagaactgcacttcagcaataatgggaacgggggcagcgttccagcctcggtttctatttataatggagacatggaaaaaatactgctggacgcacagcatgagtctggacggagtagctccaagagctctcactgtgacagtctgaggaagatgatattgaaagaaggaaagaagttgaaagcatct

SEQ ID NO: 2 - protein, 81 aa, Homo sapiens
MSQNGAPGMQEESLQGSWVELHFSNNGNGGSVPASVSIYNGDMEKILLDAQHESGRSSSKSSHCDSLRKMILKEGKKLKAS

SEQ ID NO: 3 - protein, 15 aa, artificial
  polypeptide having Bnip3 antagonist activity
LRKMILKEGKKLKAS

SEQ ID NO: 4 - protein, 194 aa, Homo sapiens
MSQNGAPGMQEESLQGSWVELHFSNNGNGGSVPASVSIYNGDMEKILLDAQHESGRSSSKSSHCDSPPRSQTPQDTNRASETDTHSIGEKNSSQSEEDDIERRKEVESILKKNSDWIWDWSSRPENIPPKEFLFKHPKRTATLSMRNTSVMKKGGIFSAEFLKVFLPSLLLSHLLAIGLGIYIGRRLTTSTSTF

SEQ ID NO: 5 - protein, 16 aa, Rattus norvegicus
SLRKIILREEEKLKVS

SEQ ID NO: 6 - DNA, 482 bp, Rattus norvegicus
atgtcgcagagcggggaggagaacctgcagggctcctgggtagaactgcacttcagcaatgggaatgggagcagcgttccagcttccgtctctatttataatggtgacatggaaaaaatactgctggatgcgcagcatgaatctggacgaagcagctccaagagctctcactgtgacagtctgaggaagattatattgagagaagaagagaagttgaaagtatcctgaagaaaaactcagattggatatgggattggtcaagtcggccagaaaatgttccccccaaggagttcctttttaaacacccgaagcgcacagctactctcagcatgagaaacacaagcgttatgaagaaagggggtattttctcagcagactttctgaaggttttccttccatctctgttactgtctcatctgttagccattggattggggatctacattggaaggcgtctgacaacttccactagtaccttttga

SEQ ID NO: 7 - protein, 75 aa, Rattus norvegicus
MSQSGEENLQGSWVELHFSNGNGSSVPASVSIYNGDMEKILLDAQHESGRSSSKSSHCDSLRKIILREEEKLKVS

SEQ ID NO: 8 - protein, 15 aa, artificial
  polypeptide having Bnip3 antagonist activity
LRKIILREEEKLKVS

SEQ ID NO: 9 - DNA, 1271 bp, Rattus norvegicus
gagcggcgcaagctgccctgctacctctcagtggtcacttcccaggcctgtcgcagttgggctccggctcctttgcggagccaccatgtcgcagagcggggaggagaacctgcagggctcctgggtagaactgcacttcagcaatgggaatgggagcagcgttccagcttccgtctctatttataatggtgacatggaaaaaatactgctggatgcgcagcatgaatctggacgaagcagctccaagagctctcactgtgacagcccacctcgctcccagacaccacaagataccaacagagctgaaatagacacccacagctttggtgagaaaaacagcactctgtctgaggaagattatattgagagaagaagagaagttgaaagtatcctaaagaaaaactcagattggatatgggattggtcaagtcggccagaaaatgttccccccaaggagttcctttttaaacacccgaagcgcacagctactctcagcatgagaaacacaagcgttatgaagaaagggggtattttctcagcagactttctgaaggttttccttccatctctgttactgtctcatctgttagccattggattggggatctacattggaaggcgtctgacaacttccactagtaccttttgatgagacttggatctgccttctgctcacacagtgaggattcacgctgagctgtgacagctaattgaagaactagcatgatccttgggtgtctgcactaagtgtgtttatttggtttggaaaagcggtgttcctgaattagggagaacgaatagactcttaccacgacctataattatacctatgggatcaattaataagcatgtcagactgatgcctattgtagcatttattagtaaatttctttgaatatattagatataaatagtataaataattttaatatagtagcaatagtgtatttaaaaatgatctgtaattgtaatccagttaagacaccttacacttcaaaagaatgacttaagtgatattaaaatcattaaagggttttccccaaaggaattctgtggccttataatcctattatgtagtagaaaattaaagggtgtgggttatttgtaaaggcctcttactttatgaattcagtagcaaggagagactaaagttacccacaaactccactttgcagtccccctcttcttgcttgcaggatgaggattccagccttgctgtccaccattaccttgggtgggcagttgggttacgcctttatctctctgctgaa

SEQ ID NO: 10 - DNA, 85 bp, artificial
  nucleotide sequence corresponding to exon 3 of a human Bnip3 mRNA
cccacctcgctcgcagacaccacaagataccaacagggcttctgaaacagatacccatagcattggagagaaaaacagctcacag

SEQ ID NO: 11 - DNA, 82 bp, artificial
  nucleotide sequence corresponding to exon3 of a rat Bnip3 mRNA
cccacctcgctcccagacaccacaagataccaacagagctgaaatagacacccacagctttggtgagaaaaacagcactctg

SEQ ID NO: 12 - DNA, 20 bp, artificial
  synthetic oligonucleotide primer
acccacagctttggtgagaa

SEQ ID NO: 13 - DNA, 23 bp, artificial
  synthetic oligonucleotide primer
cgcttgtgtttctcatgatgctg

SEQ ID NO: 14 - DNA, 19 bp, artificial
  synthetic oligonucleotide primer
ctgtgacagtctgaggaag

SEQ ID NO: 15 - DNA, 19 bp, artificial
  synthetic oligonucleotide primer
tgtttctcatgctgagagt

SEQ ID NO: 16 - DNA, 45 bp, artificial
  nucleotide sequence encoding a polypeptide having Bnip3 antagonist activity
ctgaggaagatgatattgaaagaaggaaagaagttgaaagcatct

SEQ ID NO: 17 - DNA, 48 bp, artificial
  nucleotide sequence encoding a polypeptide having Bnip3 antagonist activity
ctgaggaagattatattgagagaagaagagaagttgaaagtatcctga

SEQ ID NO: 18 - DNA, 20 bp, artificial
  synthetic oligonucleotide primer
acccacagctttggtgagaa

SEQ ID NO: 19 - DNA, 23 bp, artificial
  synthetic oligonucleotide primer
cgcttgtgtttctcatgatgctg

SEQ ID NO: 20 - DNA, 19 bp, artificial
  synthetic oligonucleotide primer
ctgtgacagtctgaggaag

SEQ ID NO: 21 - DNA, 19 bp, artificial
  synthetic oligonucleotide primer
tgtttctcatgctgagagt

SEQ ID NO: 22 - DNA, 47 bp, artificial
  short hairpin RNA oligonucleotide targeting full length Bnip3
caccgacacaagataccaacagcgaactgttggtatcttgtggtgtc

SEQ ID NO: 23 - DNA, 21 bp, artificial
  short hairpin RNA oligonucleotide targeting Bnip3 with an exon 3 deletion
cacugugacagucugaggauu

SEQ ID NO: 24 - DNA, 21 bp, artificial
  small interfering RNA oligonucleotide targeting Bnip3 with an exon 3 deletion
cacugugacagucugaggauu

SEQ ID NO: 25 - protein, 5 aa, Rattus norvegicus
LRKII

SEQ ID NO: 26 - protein, 5 aa, Rattus norvegicus
EENLQ

SEQ ID NO: 27 - protein, 5 aa, Rattus norvegicus
LHFSN

SEQ ID NO: 28 - protein, 5 aa, Rattus norvegicus
GNGSS

SEQ ID NO: 29 - protein, 5 aa, Rattus norvegicus
LLDAQ

(19) United States
(12) Patent Application Publication
(10) Pub. No.: US 2013/0196922 A1
(43) Pub. Date: Aug. 1, 2013

(54) BNIP3 ISOFORMS AND METHODS OF USE

(71) Applicant: Lorrie A. Kirshenbaum, Winnipeg (CA)

(72) Inventor: Lorrie A. Kirshenbaum, Winnipeg (CA)

(73) Assignee: UNIVERSITY OF MANITOBA, Winnipeg, MB (CA)

(21) Appl. No.: 13/811,048

(22) PCT Filed: Jul. 18, 2011
(86) PCT No.: PCT/IB2011/001662

§ 371 Date: Mar. 26, 2013

Related U.S. Application Data
(60) Provisional application No. 61/366,451, filed on Jul. 21, 2010.
(60) Provisional application No. 61/446,359, filed on Feb. 24, 2011.

(51) Int. Cl. C07K 14/47; G01N 33/68; C12N 15/113; C12Q 1/68; C07K 7/08; C07K 16/18

(52) U.S. Cl. CPC C07K 14/4747; C07K 7/08; C07K 16/18; C12N 15/113; C12Q 1/686; G01N 33/68
USPC 514/16.4; 514/44 R; 530/324; 514/21.3; 530/326; 536/23.5; 530/387.9; 514/18.9; 435/375; 435/455; 514/17.7; 435/320.1; 536/24.5; 514/44 A; 435/6.12; 435/7.1; 435/6.13

(57) ABSTRACT

The present invention provides isolated splice variants of Bnip3 and isolated polynucleotides encoding the splice variants. Also provided are isolated polypeptides present at the carboxy-terminus of Bnip3 splice variants, and antibody that specifically binds Bnip3 splice variants and/or the carboxy-terminus of Bnip3 splice variants. The present invention further provides methods of altering cellular apoptosis, cellular necrosis, cellular autophagy, or the combination thereof. For instance, the methods may include changing the amount of a Bnip3 splice variant or a carboxy-terminal region of a Bnip3 splice variant in a cell. Also provided herein are methods for determining whether death of cells in a tissue can be altered, methods for evaluating treatment options for a subject, and methods for identifying a compound that alters the amount or activity of a Bnip3 splice variant in a cell.

### CROSS-REFERENCE TO RELATED APPLICATIONS

This application claims the benefit of U.S. Provisional Application Ser. No. 61/366,451, filed Jul. 21, 2010, and U.S. Provisional Application Ser. No. 61/446,359, filed Feb. 24, 2011, each of which is incorporated by reference herein.

### BACKGROUND

Loss of cardiac cells by programmed cell death has been posited as a central underlying cause of ventricular remodeling and the decline in cardiac performance following myocardial ischemic injury (Foo et al., 2005. J. Clin. Invest 115:565-571). For this reason, there has been considerable effort over the last decade in deciphering the signaling pathways and cellular targets that govern cardiac cell death under normal and pathological conditions. Though the molecular signaling events remain poorly defined, there is considerable evidence that the mitochondrion is a conduit for integrating signals for apoptosis, necrosis, and autophagy (Baines, 2010, Annu. Rev. Physiol., 72:61-80). Several lines of investigation have implicated certain members of the Bcl-2 gene family as critical regulators of the permeability transition pore formation, cytochrome c release, and cell death (Reed, 1995, Toxicol. Lett. 82-83:155-8:155-158, Reed, et al., 1998, Biochim. Biophys. Acta, 1366:127-137, Adachi et al., 1997,1 Biol. Chem., 272:21878-21882, Crow et al., 2004, Circ. Res., 95:957-970, and Jacobson et al., 1994, Biochem. Soc. Trans., 22:600-602). Notably, the proteins Bnip3 (for Bcl-2 Nineteen Kilodalton Interacting Protein) and Nix/Bnip3L (for Bcl-2 Nineteen Kilodalton Interacting like protein X) are a subclass of evolutionary conserved BH3-domain-like members of the Bcl-2 gene family that provoke mitochondrial perturbations and cell death in response to distinct biological stresses (Regula et al., 2002, Circ. Res., 91:226-231, Yussman et al., 2002, Nat. Med., 8:725-730, Chen et al., 1999, J. Biol. Chem. 274:7-10, and Dorn and Kirshenbaum, 2008, Oncogene 27 Suppl 1:S158-S167). Previously, it was established that the carboxyl-terminal transmembrane domain of Bnip3 and Nix are crucial for insertion into mitochondrial membranes and cell death (Regula et al., 2002, Circ. Res., 91:226-231). Despite the ability of Bnip3 and Nix proteins to trigger mitochondrial perturbations, they are transcriptionally activated under different physiological conditions. Indeed, Bnip3 is induced in post-natal ventricular myocytes during hypoxia (Regula et al., 2002, Circ. Res., 91:226-231, Bruick, 2000, Proc. Natl. Acad. Sci. U.S.A, 97:9082-9087) whereas, Nix is selectively activated by Gq-signaling in response to pathological hypertrophy (Yussman et al., 2002, Nat. Med, 8:725-730, Diwan et al., 2007, J. Clin. Invest., 117:2825-2833). Despite their overlapping ability to disrupt mitochondrial function in different cardiac pathologies, the biological significance of Bnip3 and Nix proteins in regulating cell death more generally is undetermined.

Previously, it was established that Bnip3 promoter activity is strongly repressed under basal normoxic conditions but is readily induced during hypoxia. The induction of Bnip3 gene transcription and cell death during hypoxia has been attributed to the displacement of inhibitory NF-κB-HDAC complexes, which relieves the steric hindrance on the Bnip3 promoter (Shaw et al., 2008, Proc. Natl. Acad. Sci. U.S.A., 105:20734-20739, Baetz et al., 2005, Circulation, 112:3777-3785). Nevertheless, despite the tight regulation of Bnip3 transcription, certain cancer cells are resistant to hypoxic injury (Demaria et al., 2010, J. Immunother., 33:335-351, Green et al., 1994, Important. Adv. Oncol., 1994:37-52, Kothari et al., 2003, Oncogene, 22:4734-4744, Bellot et al., 2009, Mol. Cell Biol., 29:2570-2581, Chiche et al., 2010, J. Cell Physiol., 222:648-657, and Mazure et al., 2010, Curr. Opin. Cell Biol., 22:177-180). While the underlying mechanisms for this apparent resistance to hypoxic stress are unknown, it likely reflects an adaptive survival mechanism to oppose the otherwise lethal actions of Bnip3 on apoptosis.

### SUMMARY OF THE INVENTION

The present invention provides a method for altering apoptosis, necrosis, autophagy, or the combination thereof, of a cell. The method may include expressing in a cell an effective amount of a polypeptide having Bnip3 antagonist activity, wherein (i) the amino acid sequence of the polypeptide and the amino acid sequence of SEQ ID NO:2 have at least 84% identity, or (ii) the amino acid sequence of the polypeptide and the amino acid sequence of SEQ ID NO:7 have at least 80% identity, wherein apoptosis, necrosis, autophagy, or the combination thereof, is altered in the cell compared to a control cell. The expressing may include, for instance, introducing the polypeptide into the cell, or introducing into the cell a polynucleotide encoding the polypeptide. The cell may be ex vivo or in vivo, and may be, for instance, a cancer cell or a cardiac cell. Apoptosis, necrosis, autophagy, or the combination thereof may be increased or decreased in the cell.

The present invention also provides an isolated polypeptide having Bnip3 antagonist activity, wherein the polypeptide include an amino acid sequence having at least 80% sequence identity to SEQ ID NO: 2 or at least 80% sequence identity to SEQ ID NO:7. The isolated polypeptide may be a fusion polypeptide. Other isolated polypeptides provided herein include, but are not limited to, a polypeptide including amino acids LRKMILKEGKKLKAS (SEQ ID NO:3), and a polypeptide comprising amino acids LRKIILREEEKLKVS (SEQ ID NO:8). In one embodiment, the present invention includes a polypeptide comprising amino acids LRKIILREEEKLKVS (SEQ ID NO:8) wherein the polypeptide includes either no greater than 102 amino acids or at least 104 amino acids. In one embodiment, the polypeptides described herein may include one or more conservative substitutions. The present invention also includes compositions including the polypeptides described herein, including compositions that further include a pharmaceutically acceptable carrier.

The present invention also provides isolated polynucleotides. In one embodiment, the polynucleotides include (a) a nucleotide sequence encoding a polypeptide having Bnip3 antagonist activity, wherein (i) the amino acid sequence of the polypeptide and the amino acid sequence of SEQ ID NO:2 have at least 80% identity, or (ii) the amino acid sequence of the polypeptide and the amino acid sequence of SEQ ID NO:7 have at least 80% identity, or (b) the full complement of the nucleotide sequence of (i) or (ii). In another embodiment, the polynucleotides include (a) a nucleotide sequence encoding a polypeptide having Bnip3 antagonist activity, wherein the polynucleotide has at least 80% identity to SEQ ID NO:1 or SEQ ID NO:6, or (b) the full complement of the nucleotide sequence of (a). In one embodiment, the polynucleotides may have between 18 and 30 nucleotides, wherein the nucleotide sequence of the isolated polynucleotide includes (a) nucleotides 197 and 198 of SEQ ID NO:1 and consecutive nucleotides selected from nucleotides 169 through 226 of SEQ ID NO:1, or the complement thereof, or (b) nucleotides selected from nucleotides 198-243 of SEQ ID NO:1, or the complement thereof. A polynucleotide described herein may include a heterologous polynucleotide, such as a regulatory sequence and/or a vector. A polynucleotide of the present invention may be DNA, RNA, or a combination thereof.

The present invention also includes antibody. In one aspect the antibody specifically binds a polypeptide that includes SEQ ID NO:2, wherein the antibody does not bind to a polypeptide comprising an amino acid sequence SEQ ID NO:4. In one aspect the antibody specifically binds a polypeptide that includes SEQ ID NO:7, wherein the antibody does not bind to a polypeptide comprising an amino acid sequence SEQ ID NO:9. The present invention also provides an antibody that specifically binds a polypeptide having LRKMILKEGKKLKAS (SEQ ID NO:3) or LRKIILREEEKLKVS (SEQ ID NO:8). The antibody may be polyclonal or monoclonal.

The present invention also provides a method of making an antibody. The method includes administering to an animal a polypeptide comprising at least 6 consecutive amino acids selected from LRKMILKEGKKLKAS (SEQ ID NO:3) or LRKIILREEEKLKVS (SEQ ID NO:8). The method may further include isolating the antibody. The present invention also includes the antibody produced by the method.

The present invention provides a method that includes administering to a subject in need thereof an effective amount of a composition, wherein apoptosis, necrosis, autophagy, or the combination thereof, is decreased in the subject. The composition may include a polynucleotide that includes a nucleotide sequence encoding a polypeptide having Bnip3 antagonist activity, wherein (i) the amino acid sequence of the polypeptide and the amino acid sequence of SEQ ID NO:2 have at least 84% identity, or (ii) the amino acid sequence of the polypeptide and the amino acid sequence of SEQ ID NO:7 have at least 80% identity, In another embodiment the composition may include a polypeptide having Bnip3 antagonist activity, wherein (i) the amino acid sequence of the polypeptide and the amino acid sequence of SEQ ID NO:2 have at least 84% identity, or (ii) the amino acid sequence of the polypeptide and the amino acid sequence of SEQ ID NO:7 have at least 80% identity. The administering may include delivery of the polynucleotide or the polypeptide to cardiac tissue or brain tissue. The subject may have signs of or is at risk of a disease chosen from acute myocardial infarction, hypoxia, myocardial ischemia, myocardial infarction, stroke, or vascular disease. The method may result in a reduction of a sign of disease.

Also provided by the present invention is a method that includes administering to a subject in need thereof an effective amount of a composition, wherein cellular apoptosis, necrotic cell death, autophagy, or the combination thereof is increased in the subject. The composition may include a polynucleotide having between 18 and 30 nucleotides, wherein the nucleotide sequence of the isolated polynucleotide includes nucleotides 197 and 198 of SEQ ID NO:1, or the complement thereof, nucleotides selected from nucleotides 198-243 of SEQ ID NO:1, or the complement thereof, or 1, or nucleotides 179 and 180 of SEQ ID NO:6, or the complements thereof. The subject may have signs or is at risk of cancer, such as a cancer selected from, for instance, pancreatic cancer, colon cancer, or breast cancer. The method may result in a reduction of a sign of disease.

Also provided herein is a method for determining whether death of cells in a tissue can be decreased. The method may include determining whether cells present in diseased tissue of a biological sample express a Bnip3Δex3 polypeptide, wherein the absence of Bnip3Δex3 polypeptide compared to a control cell indicates that death of the cells in the diseased tissue can be decreased. The method may further include obtaining a biological sample from a subject, wherein the biological sample includes diseased tissue. The determining may include use of an antibody that specifically binds SEQ ID NO:3 and/or SEQ ID NO:8. The determining may include use of a PCR assay. The method may further include administering to the subject an effective amount of a composition. The composition may include a polynucleotide including a nucleotide sequence encoding a polypeptide having Bnip3 antagonist activity, wherein (i) the amino acid sequence of the polypeptide and the amino acid sequence of SEQ ID NO:2 have at least 80% identity, or (ii) the amino acid sequence of the polypeptide and the amino acid sequence of SEQ ID NO:7 have at least 80% identity. In another embodiment, the composition may include a polypeptide having Bnip3 antagonist activity, wherein (i) the amino acid sequence of the polypeptide and the amino acid sequence of SEQ ID NO:2 have at least 80% identity, or (ii) the amino acid sequence of the polypeptide and the amino acid sequence of SEQ ID NO:7 have at least 80% identity. The method may include delivery of the polynucleotide or the polypeptide to cardiac tissue or brain tissue. The subject may have signs of or is at risk of a disease chosen from acute myocardial infarction, hypoxia, myocardial ischemia, myocardial infarction, stroke, or vascular disease. The method may result in a reduction of a sign of disease.

The present invention provides a method for determining whether death of cells in a tissue can be increased. The method may include determining whether cells present in diseased tissue of a biological sample express a Bnip3Δex3 polypeptide, wherein the presence of Bnip3Δex3 polypeptide indicates that death of the cells in the diseased tissue can be increased. The method may further include obtaining a biological sample from a subject, wherein the biological sample comprises diseased tissue. In one embodiment, The determining may include use of an antibody that specifically binds SEQ ID NO:3 and/or SEQ ID NO:8. In one embodiment, the determining includes use of a PCR assay. The method may further include administering to the subject an effective amount of a composition. The composition may include a polynucleotide including between 18 and 30 nucleotides, wherein the nucleotide sequence of the isolated polynucleotide includes nucleotides 197 and 198 of SEQ ID NO:1, or the complement thereof, nucleotides selected from nucleotides 198-243 of SEQ ID NO:1, or the complement thereof, or nucleotides 179 and 180 of SEQ ID NO:6, or the complements thereof. The diseased tissue may include cancer tissue, such as cancer tissue selected from pancreatic cancer, colon cancer, or breast cancer. The method may result in a reduction of a sign of disease.

The present invention also provides a method for evaluating treatment options for a subject. The method may include determining whether cells present in a diseased tissue of a biological sample express a Bnip3Δex3 polypeptide, wherein the absence of Bnip3Δex3 polypeptide compared to a control cell indicates that death of the cells in the diseased tissue can be decreased, and wherein the presence of Bnip3Δex3 polypeptide indicates that death of the cells in the diseased tissue can be increased. The method may further include obtaining a biological sample from the subject, wherein the biological sample include diseased tissue. The determining may include use of an antibody that specifically binds SEQ ID NO:3 and/or SEQ ID NO:8. The determining may include use of a PCR assay.

Also provided by the present invention is a method for identifying a compound that alters the amount or activity of a Bnip3Δex3 polypeptide in a cell. The method may include exposing a cell to a compound, and measuring the amount of Bnip3Δex3 polypeptide in the cell, the activity of Bnip3Δex3 polypeptide in the cell, or the combination thereof, wherein a change in the amount of Bnip3Δex3 polypeptide in the cell, the activity of Bnip3Δex3 polypeptide in the cell, or the combination thereof, compared to a control cell not exposed to the compound indicates the compound alters the amount or activity of Bnip3Δex3 polypeptide in the cell. The amount or activity of Bnip3Δex3 polypeptide in the cell may be increased or decreased.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

As used herein, “gene” refers to a nucleotide sequence that encodes an mRNA. A gene has at its 5′ end a transcription initiation site and a transcription terminator at its 3′ end. As used herein, a “target gene” refers to a specific gene whose expression is inhibited by a polynucleotide as described herein. As used herein, a “target mRNA” is an mRNA encoded by a target gene. Unless noted otherwise, a target gene can result in multiple mRNAs distinguished by the use of different combinations of exons. Such related mRNAs are referred to as splice variants or transcript variants of a gene.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. For instance, a coding region present in a wild-type microbe and encoding a polypeptide is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide of the present invention through the use of standard genetic and/or recombinant methodologies well known to one skilled in the art. A polynucleotide of the present invention may be included in a suitable vector. The presence of heterologous nucleotides flanking one or both sides of a polynucleotide described herein result from human manipulation.

The terms “complement” and “complementary” as used herein, refer to the ability of two single stranded polynucleotides to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The term “substantial complement” and cognates thereof as used herein, refer to a polynucleotide that is capable of selectively hybridizing to a specified polynucleotide under stringent hybridization conditions. Stringent hybridization can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically substantially complementary to a second polynucleotide if hybridization occurs between the polynucleotide and the second polynucleotide. As used herein, “specific hybridization” refers to hybridization between two polynucleotides under stringent hybridization conditions.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, enzyme, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. For instance, a coding region present in a wild-type microbe and encoding a polypeptide is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide of the present invention through the use of standard genetic and/or recombinant methodologies well known to one skilled in the art. A polynucleotide of the present invention may be included in a suitable vector. The presence of heterologous nucleotides flanking one or both sides of a polynucleotide described herein result from human manipulation.

As used herein, “Bnip3 antagonist activity” refers to activity of Bnip3Δex3 polypeptides described herein. A polypeptide having Bnip3 antagonist activity will interact with a Bnip3 polypeptide, will suppress hypoxia-induced loss of mitochondrial ΔΨm in cells, will suppress hypoxia-induced ROS production in cells, and will suppress hypoxia-induced cell death. Methods for determining whether a polypeptide has Bnip3 antagonist activity are described herein and include the use of, for instance, hypoxic cardiac myocytes and hypoxic pancreatic cancer cells. Assays for determining whether a polypeptide has Bnip3 antagonist activity may be determined by in vitro and in vivo assays.

As used herein, an “isolated” substance is one that has been removed from its natural environment, produced using recombinant techniques, or chemically or enzymatically synthesized. For instance, a polypeptide or a polynucleotide can be isolated. Preferably, a substance is purified, i.e., is at least 60% free, preferably at least 75% free, and most preferably at least 90% free from other components with which they are naturally associated.

As used herein, an antibody that can “specifically bind” or is “specific for” a polypeptide is an antibody that interacts only with an epitope of the antigen that induced the synthesis of the antibody, or interacts with a structurally related epitope.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The summary of the present invention presented above is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Hypoxia-Induced Alternative Splicing of Bnip3 in Ventricular Myocytes in vitro and in vivo. Panel A: Schematic representation depicting Bnip3FL and alternative spliced isoform Bnip3Δex3. Panel B: Radioisotope P-labeled semi-quantitative RT-PCR of hypoxia-induced splicing of endogenous Bnip3 gene in post-natal ventricular myocytes. Ventricular myocytes were isolated and subjected to hypoxia conditions for 24 hours. RNA were extracted and analyzed by semi-quantitive RT-PCR with P-labelled primer. An extra 482 nt splicing product was detected only under hypoxic conditions by denaturing PAGE. Panel C: Western blot analysis of Bnip3 proteins in ventricular myocytes. Cell lysates from ventricular myocytes infected by adenovirous encoding His-vector, His-Bnip3FL or His-Bnip3Δex3 were analyzed by western blot. The filter was probed with a murine antibody directed against His tag. Panel D & E: Relative mRNA expression levels of Bnip3FL (Panel D) and Bnip3Δex3 (Panel E) in post-natal ventricular myocytes assessed by real time PCR. Time and hypoxia severity dependent induction of Bnip3FL and Bnip3Δex3 mRNA were detected in ventricular myocytes. Panel F: Real time PCR analysis of Bnip3FL and Bnip3Δex3 isoforms in adult hearts following myocardial infarction in vivo. Bnip3FL and Bnip3Δex3 were analyzed in sham operated control adult hearts and myocardial infarcted hearts following 24 hour coronary artery ligation by real time PCR. Panel G: Bnip3FL and Bnip3Δex3 gene transcription determined by real time PCR in ventricular myocytes defective for NF-κB signaling with the non-phosphorylatable mutant of IκBa (IκBsa). Post-natal ventricular myocytes were infected with adenovirus encoding empty vector control or IκBsa. *=Statistically different from CTRL. CTRL: control; HPX: hypoxia; FL: full length.

FIG. 2. Bnip3Δex3 Suppresses Mitochondrial Perturbations and Cell Death Induced by Bnip3FL. Panel A: Localization of Bnip3 proteins in ventricular myocytes. Epifluorescence microscopy of post-natal ventricular myocytes expressing plasmids encoding GFP-Bnip3FL or GFP-Bnip3Δex3 proteins in the presence of Mitotracker Red to visualize mitochondria (Red) or Hoechst to visualize nucleus (Blue). Panel B: Mitochondrial membrane potential in the presence and absence of Bnip3 proteins. Mitochondrial Δψm was monitored by TMRM staining of ventricular myocytes in the presence and absence of Bnip3FL and Bnip3Δex3. Panel C: Reactive oxygen species (ROS) in ventricular myocytes. ROS production was monitored by dihydroethidium (DHE) staining in the presence and absence of Bnip3FL or Bnip3Δex3. Panel D: Western blot analysis depicting interaction of Bnip3FL and Bnip3Δex3. 500 μg cell lysates were immunoprecipitated with murine antibodies directed against GFP Tags. Bound proteins were detected with the antibodies directed against GFP or HA tag. Panel E: Western blot analysis of mitochondrial and cytoplasmic fractions from cells analyzed in the absence or presence of Bnip3FL and Bnip3Δex3. The filter was probed with a murine antibody directed against mitochondrial protein VDAC1 to verify the integrity and purity of the cell fractionation. Bnip3FL was detected with a murine antibody directed against Bnip3. GFP-vector and GFP-Bnip3Δex3 were detected with the antibody directed against GFP tag. Panel F: Cell viability of ventricular myocytes. Representative epifluorescence images of cells stained with vital dyes calcein AM and ethidium homodimer to visualize live (Green) and dead (Red) cells, respectively. Panel G: Histogram depicts quantitative data from panel F for repression of Bnip3FL induced cell death by Bnip3Δex3 in a dose dependent manner. Bnip3FL adenovirus (1×10 ifu/ml) was used for all conditions tested. Data was obtained from at least n=3-4 independent myocyte isolations counting >500 cells from n=3 glass coverslips for each condition tested. *=Statistically different from CTRL; †=Statistically different from Bnip3FL.

FIG. 3. Effects of Bnip3Δex3 on Cell Viability during Hypoxia. Panel A: Strategy of knocking down endogenous Bnip3FL in ventricular myocytes. Short hairpin interference RNA was designed against full length Bnip3 (shRNA-BnipFL) to test the effects of Bnip3Δex3 isoform on cell viability in the absence of the Bnip3FL. Panels (B, C, D): Western blot or Real time PCR analysis of Bnip3FL and Bnip3Δex3 levels in ventricular myocytes. Postnatal ventricular myocytes were treated with shRNA-Bnip3FL under normoxic and hypoxic conditions. Panels (E, F, G): Effects of Bnip3FL Knock-down on Mitochondrial Membrane Potential and Cell Viability. E: Neonatal ventricular myocytes were assessed for mitochondrial membrane potential Δψm by TMRM staining in the absence or presence of shRNA-Bnip3FL or Bnip3Δex3 under normoxic and hypoxic conditions. F: Cell viability of ventricular myocytes for conditions in Panel E. Representative images of cells stained with vital dyes calcein AM and ethidium homodimer to visualize the number of live (Green) and dead (Red) cells, respectively. G: Quantitative data for Panel F. Data was obtained from at least n=3-4 independent myocyte isolations counting >200 cells from n=3 glass coverslips for each condition tested. *=Statistically significant; NS=Not statistically significant; †=Statistically different from HPX.

FIG. 4. Bnip3Δex3 Promotes Survival of Ventricular Myocytes. Panel A: Strategy to knock-down endogenous Bnip3Δex3 isoform. siRNA-Bnip3Δex3 was designed to specifically target sequences spanning the exon2-exon4 junction which is only present in Bnip3Δex3 isoform. Panels (B, C): Real time PCR analysis of relative mRNA levels of endogenous Bnip3FL and Bnip3Δex3. Effects of hypoxia on ventricular myocytes in the absence or presence of siRNA-Bnip3Δex3 were determined by real time PCR. Panel D: Cell viability of ventricular myocytes following Bnip3Δex3 knock-down with siRNA-Bnip3Δex3 (50 nM and 100 nM) under normoxic and hypoxic conditions. Representative epifluorescence images of cells stained with vital dyes calcein AM and ethidium homodimer to visualize live (green) and dead (red) cells, respectively. Panel E: Histogram for quantitative data shown in Panel D. Panel F: Cell viability of Bnip3-/-MEF cells reconstituted with Bnip3FL in the absence or presence of Bnip3Δex3 were analyzed by epifluorescence microscopy. Representative images of cells stained with vital dyes calcein-AM and ethidium homodimer-1 to visualize living (green) and dead (red) cells, respectively. Panel G: Histogram for quantitative data shown in Panel F. Data was obtained from at least n=3-4 independent myocyte isolations counting >200 cells from n=3 glass coverslips for each condition tested. NS=Not statistically significant; *=Statistically significant; †=Statistically different from HPX; **=Statistically different from Bnip3FL.

FIG. 5. Model for the regulation of hypoxia-induced alternatively spliced variant Bnip3Δex3 and cell survival. Hypoxia induces constitutive Bnip3FL mRNA with exon1-exon6 which encodes Bnip3FL protein that contains the carboxyl terminal transmembrane domain (TM), provokes mitochondrial defects and cell death (arrows on left side of figure). Hypoxia-induced alternatively spliced variant Bnip3Δex3 (missing exon3) encodes a truncated Bnip3 proteins lacking the putative BH3 and carboxyl terminal transmembrane domains that promotes survival by antagonizing Bnip3FL (arrows on right side of figure); Mitochondrial membrane potential (Δψm); Reactive oxygen species (ROS).

FIG. 6. Reactive oxygen species (ROS) in ventricular myocytes. Postnantal ventricular myocytes were infected with Bnip3FL or Bnip3Δex3 and assessed for ROS production by fluorescence microscopy using 10 μM CM-H2DCFDA as a ROS probe.

FIG. 7. Flow cytometry analysis of ventricular myocytes. Ventricular myocytes were subject to hypoxia in the absence and presence of Bnip3Δex3. Flow cytometric dot plots are shown based on cell staining with Annexin V and DAPI. The proportions of events (%) were labelled in the quadrants representing live cells (Annexin V− DAPI−), early apoptotic cells (Annexin V+ DAPI−) or late apoptotic/necrotic cells (Annexin V+ DAPI+).

FIG. 8. A comparison of SEQ ID NO:2 (a human Bnip3Δex3 polypeptide) and SEQ ID NO:7 (a rat Bnip3Δex3 polypeptide). Identical are marked with “*” and conserved amino acids are marked “:”.

FIG. 9. Nucleotide (SEQ ID NO:1) and amino acid sequence (SEQ ID NO:2) of a human Bnip3Δex3 polypeptide, a wild-type Bnip3Δex3 polypeptide (SEQ ID NO:4), nucleotide (SEQ ID NO:6) and amino acid sequence (SEQ ID NO:7) of a rat Bnip3Δex3 polypeptide, and a wild-type rat Bnip3Δex3 polypeptide (SEQ ID NO:9).

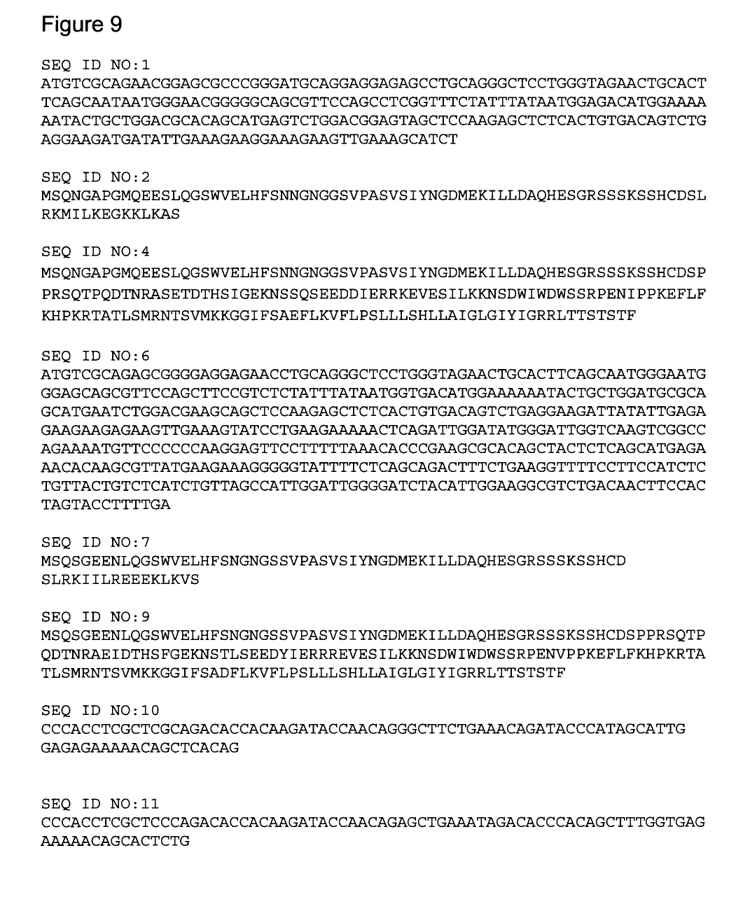

FIG. 10. Real time PCR analysis of Bnip3FL and Bnip3Δex3 isoforms in adult hearts following aortic banding in vivo. Bnip3FL and Bnip3Δex3 were analyzed in control adult hearts and failing hearts following 3 weeks of aortic banding by real time PCR. HF: heart failure. Heart failure was induced by pressure over load by aortic banding. Neither Bnip3 or Bnip3Δex3 were changed during heart failure.

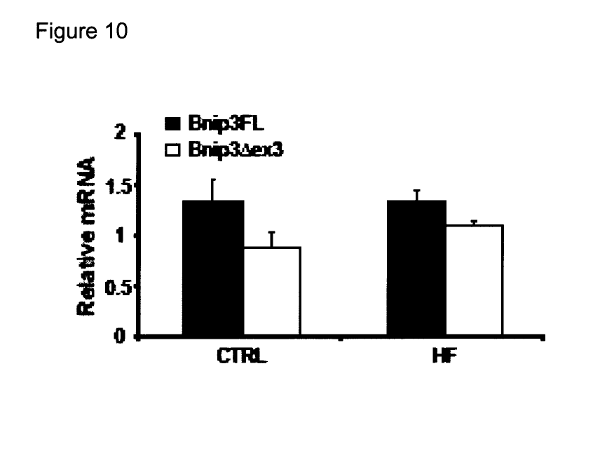

### DETAILED DESCRIPTION OF ILLUSTRATIVE EMBODIMENTS

Bnip3 polypeptides are known in the art (see, for instance, GenBank NP_{—}004043 and GenBank AF243515 for examples of human and rat Bnip3 polypeptides, respectively), and provoke mitochondrial perturbations and cell death in response to distinct biological stresses. A Bnip3 polypeptide is encoded by a gene containing 6 exons, and the 6 exons are translated to result in a Bnip3 polypeptide. The inventors have identified a Bnip3 splice variant, referred to herein as a Bnip3Δex3 polypeptide, that does not include the amino acids encoded by exon 3. The splicing of exons 2 and 4 shifts the reading frame, causing translation of the mRNA to yield a different series of amino acids beginning after the junction of exons 2 and 4. The Bnip3 splice variant (molecular weight of 8.2 kDa) is shorter than the wild-type Bnip3 polypeptide (molecular weight of 26 kDa) due to a premature stop codon located in exon 4. The inventors have also found that the Bnip3 splice variant inhibits the cytotoxic action of Bnip3, and targets the Bnip3 splice variant to the endoplasmic reticulum.

The present invention includes isolated polypeptides. In one embodiment, a polypeptide has Bnip3 antagonist activity. A polypeptide having Bnip3 antagonist activity is referred to herein as a Bnip3Δex3 polypeptide. Examples of Bnip3Δex3 polypeptides are depicted at SEQ ID NO:2 and SEQ ID NO:7. Other examples of Bnip3Δex3 polypeptides include those that are structurally similar to the amino acid sequence of SEQ ID NO:2 or SEQ ID NO:7. A Bnip3Δex3 polypeptide that is structurally similar to the amino acid sequence of SEQ ID NO:2 or SEQ ID NO:7 has Bnip3 antagonist activity. A Bnip3Δex3 polypeptide has a molecular weight of 8.2 kDa.

Structural similarity of two polypeptides can be determined by aligning the residues of the two polypeptides (for example, a candidate polypeptide and SEQ ID NO:2 or SEQ ID NO:7, or a candidate polypeptide and SEQ ID NO:3 or SEQ ID NO:8, both of which are described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference polypeptide may be a polypeptide described herein. A candidate polypeptide is the polypeptide being compared to the reference polypeptide. A candidate polypeptide may be isolated, for example, from a cell of an animal, such as a human, rat, or mouse, or can be produced using recombinant techniques, or chemically or enzymatically synthesized.

Unless modified as otherwise described herein, a pair-wise comparison analysis of amino acid sequences can be carried out using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, polypeptides may be compared using the BESTFIT algorithm in the GCG package (version 10.2, Madison Wis.).

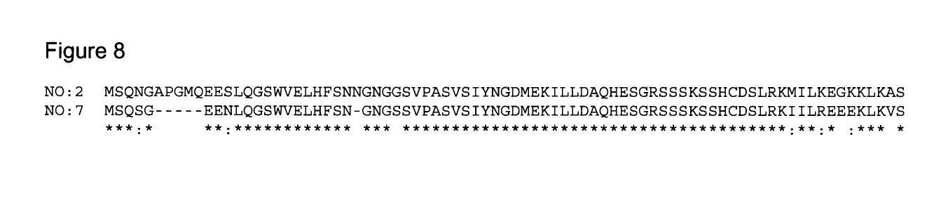

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. FIG. 8 shows a comparison of SEQ ID NO:2 (human Bnip3Δex3 polypeptide) and SEQ ID NO:7 (rat Bnip3Δex3 polypeptide). Identical amino acids are marked with “*” and conserved amino acids are marked “:”.

Deletion of the carboxy terminal 16 amino acids (SLRKIILREEEKLKVS, SEQ ID NO:5) of the Bnip3Δex3 depicted at SEQ ID NO:7 abrogated its ability to co-localize with the endoplasmic reticulum and its ability to suppress hypoxia-induced cell death. However, deletion of amino acids 61-65 (LRKII (SEQ ID NO:_), amino acids present in the C-terminal polypeptide) had no effect on the ability of Bnip3Δex3 to suppress hypoxia-induced loss of cell viability. Other mutations of Bnip3Δex3 also had no effect on the ability of Bnip3Δex3 to suppress hypoxia-induced loss of cell viability. Bnip3Δex3 (SEQ ID NO:7) mutants containing a deletion of amino acids 6-10 (EENLQ, SEQ ID NO:_), a deletion of amino acids 16-20 (LHFSN, SEQ ID NO:_), a deletion of amino acids 21-25 (GNGSS, SEQ ID NO:_), or a deletion of amino acids 41-45 (LLDAQ, SEQ ID NO:_) were able to suppress hypoxia-induced loss cell death.

Thus, as used herein, a Bnip3Δex3 polypeptide of the present invention includes those with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to SEQ ID NO:2 or SEQ ID NO:7.

Alternatively, as used herein, a Bnip3Δex3 polypeptide of the present invention includes those with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to SEQ ID NO:2 or SEQ ID NO:7.

Whether a candidate polypeptide has Bnip3 antagonist activity may be determined by in vitro or in vivo assays. One assay evaluates the ability of a polypeptide to interact with a Bnip3 polypeptide. Standard immunoprecipitation assays may be used. The antibody used may be antibody that specifically binds a Bnip3 polypeptide such as SEQ ID NO:2 or SEQ ID NO:7. Alternatively, when a candidate polypeptide or a Bnip3 polypeptide is a fusion polypeptide with an additional amino acid sequence such as, for instance, GFP or a His-tag, antibody specific to the additional amino acid sequence may be used. A candidate polypeptide that is structurally similar to a Bnip3Δex3 polypeptide and interacts with a Bnip3 polypeptide has Bnip3 antagonist activity.

Another assay that can be used to determine whether a candidate polypeptide has Bnip3 antagonist activity is whether the candidate polypeptide will suppress hypoxia-induced loss of mitochondrial ΔΨm in cells, and/or suppress hypoxia-induced ROS production in cells. In one embodiment, the types of cells that may be used in such an assay are those that undergo apoptosis when exposed to hypoxic conditions. Examples of such cells include, but are not limited to, cardiac cells such as post-natal ventricular myocytes, pancreatic cancer cells, human embryonic kidney cells, breast cancer cells, and human colorectal cancer cells. The cells are transfected with an expression vector that encodes and expresses the candidate polypeptide under hypoxic conditions, and changes in the mitochondrial ΔΨPm of the cells are compared to control cells as described in Example 1. When a cell that includes a candidate polypeptide displays little to no change in mitochondrial ΔΨm under the same conditions where the control cell displays a significant reduction in mitochondrial activity, then the candidate polypeptide has Bnip3 antagonist activity. Alternatively, changes in the ROS production by cells transfected with the expression vector are compared to control cells as described in Example 1. When a cell that includes a candidate polypeptide displays little to no change in ROS production under the same conditions where the control cell displays a significant increase in ROS production, then the candidate polypeptide has Bnip3 antagonist activity.

As discussed above, the inventors have identified a splice variant of a Bnip3 polypeptide that does not include the amino acids encoded by exon 3 and instead includes a series of amino acids not present in Bnip polypeptide resulting from exons 1-6. In another embodiment, a polypeptide of the present invention includes a polypeptide that is present in a Bnip3Δex3 polypeptide and not present in a Bnip3 polypeptide. Such a polypeptide is referred to herein as a “C-terminal” polypeptide. Examples of C-terminal polypeptides include, but are not limited to, LRKMILKEGKKLKAS (SEQ ID NO:3) and LRKIILREEEKLKVS (SEQ ID NO:8). In one embodiment a C-terminal polypeptide includes at least one additional amino acid at the amino-terminal end, and in one embodiment the additional amino acid is a serine. Other examples of C-terminal polypeptides include those that are structurally similar to the amino acid sequence of SEQ ID NO:3 or SEQ ID NO:8. In one embodiment, a C-terminal polypeptide has activity, such as the ability to target the endoplasmic reticulum. Methods for determining whether a C-terminal polypeptide described herein targets endoplasmic reticulum in a cell are known to the skilled person and are routine. For instance, a fusion between a C-terminal polypeptide described herein and a fluorescent polypeptide may be expressed in a cell and the location of the fluorescent polypeptide monitored in the cell using conventional methods. FIG. 8 shows a comparison of SEQ ID NO:2 (human Bnip3Δex3 polypeptide) and SEQ ID NO:7 (rat Bnip3Δex3 polypeptide), where SEQ ID NO:3 corresponds to amino acids 67-81 of SEQ ID NO:2 in FIG. 8, and SEQ ID NO:8 corresponds to amino acids 61-75 of SEQ ID NO:7 in FIG. 8. Identical amino acids are marked with “*” and conserved amino acids are marked “:”.

Thus, as used herein, a C-terminal polypeptide of the present invention includes those with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to SEQ ID NO:3 or SEQ ID NO:8.

Alternatively, as used herein, a C-terminal polypeptide of the present invention includes those with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to SEQ ID NO:3 or SEQ ID NO:8.

In one embodiment, the present invention also includes C-terminal polypeptides that have at least 6, at least 7, at least 8, at least 9, at least 10, at least 11, at least 12, at least 13, at least 14, or at least 15 consecutive amino acids, for instance, at least 6 to at least 15 consecutive amino acids from SEQ ID NO:3 or SEQ ID NO:8. In one embodiment, the present invention also includes C-terminal polypeptides that include the amino acid sequence of a C-terminal polypeptide, such as SEQ ID NO:3 or SEQ ID NO:8, and further include at least 1, at least 2, at least 3, at least 4, or at least 5 conservative substitutions. In one embodiment, the present invention also includes C-terminal polypeptides that include the amino acid sequence of a C-terminal polypeptide, such as SEQ ID NO:3 or SEQ ID NO:8, and no greater than 98, no greater than 99, no greater than 100, no greater than 101, no greater than 102, or no greater than 103 additional amino acids, and in one embodiment, the present invention also includes C-terminal polypeptides that include the amino acid sequence of a C-terminal polypeptide, such as SEQ ID NO:3 or SEQ ID NO:8, and at least 104, at least 105, at least 106, at least 107, at least 108 additional amino acids. A C-terminal polypeptide may be produced using recombinant techniques, or chemically or enzymatically synthesized using routine methods.

A polypeptide of the present invention may be expressed as a fusion that includes an additional amino acid sequence not normally or naturally associated with the polypeptide. In one embodiment, the additional amino acid sequence may be useful for purification of the fusion polypeptide by affinity chromatography. Various methods are available for the addition of such affinity purification moieties to proteins. Representative examples include, for instance, polyhistidine-tag (His-tag) and maltose-binding protein (see, for instance, Hopp et al. (U.S. Pat. No. 4,703,004), Hopp et al. (U.S. Pat. No. 4,782,137), Sgarlato (U.S. Pat. No. 5,935,824), and Sharma (U.S. Pat. No. 5,594,115)). In one embodiment, the additional amino acid sequence may be a carrier polypeptide. The carrier polypeptide may be used to increase the immunogenicity of the fusion polypeptide to increase production of antibodies that specifically bind to a polypeptide of the invention. The invention is not limited by the types of carrier polypeptides that may be used to create fusion polypeptides. Examples of carrier polypeptides include, but are not limited to, keyhole limpet hemacyanin, bovine serum albumin, ovalbumin, mouse serum albumin, rabbit serum albumin, and the like. In another embodiment, the additional amino acid sequence may be a fluorescent polypeptide (e.g., green, yellow, blue, or red fluorescent proteins) or other amino acid sequences that can be detected in a cell, for instance, a cultured cell, or a tissue sample that has been removed from an animal. In one embodiment, the additional amino acid sequence may be targeted to the endoplasmic reticulum. For example, a C-terminal polypeptide of the present invention may target the endoplasmic reticulum, and an amino acid sequence fused to a C-terminal polypeptide described herein would likewise be targeted to the endoplasmic reticulum.

The present invention also includes isolated polynucleotides encoding a Bnip3Δex3 polypeptide of the present invention. A polynucleotide encoding a Bnip3Δex3 polypeptide is referred to herein as Bnip3Δex3 polynucleotide. Bnip3Δex3 polynucleotides may have a nucleotide sequence encoding a polypeptide having the amino acid sequence shown in SEQ ID NO:2 or SEQ ID NO:7. An example of the class of nucleotide sequences encoding such a polypeptide is SEQ ID NO:1 and SEQ ID NO:6, respectively. It should be understood that a polynucleotide encoding a Bnip3Δex3 polypeptide represented by SEQ ID NO:2 or SEQ ID NO:7 is not limited to the nucleotide sequence disclosed at SEQ ID NO:1 or SEQ ID NO:6, but also includes the class of polynucleotides encoding such polypeptides as a result of the degeneracy of the genetic code. For example, the naturally occurring SEQ ID NO:1 is but one member of the class of nucleotide sequences encoding a polypeptide having the amino acid sequence SEQ ID NO:2. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

A Bnip3Δex3 polynucleotide of the present invention may have sequence similarity with the nucleotide sequence of SEQ ID NO:1 or SEQ ID NO:6. Bnip3Δex3 polynucleotides having sequence similarity with the nucleotide sequence of SEQ ID NO:1 or SEQ ID NO:6 encode a Bnip3Δex3 polypeptide. A Bnip3Δex3 polynucleotide may be isolated from a cell, such as a human cell or a rat cell, or may be produced using recombinant techniques, or chemically or enzymatically synthesized. A Bnip3Δex3 polynucleotide of the present invention may further include heterologous nucleotides flanking the open reading frame encoding the Bnip3Δex3 polynucleotide. Typically, heterologous nucleotides may be at the 5′ end of the coding region, at the 3′ end of the coding region, or the combination thereof. The number of heterologous nucleotides may be, for instance, at least 10, at least 100, or at least 1000.

The present invention also includes fragments of the polypeptides described herein, and the polynucleotides encoding such fragments, Bnip3Δex3 polypeptides (such as SEQ ID NO:2 and SEQ ID NO:6), as well as those polypeptides having structural similarity to SEQ ID NO:2 or SEQ ID NO:6. A polypeptide fragment may include a sequence of at least 30, at least 35, at least 40, at least 45, at least 50, at least 55, at least 60, at least 65, at least 70, at least 71, at least 72, at least 73, at least 74, at least 75, at least 76, at least 77, at least 78, at least 79, or at least 80 amino acid residues.

The present invention also includes polynucleotides that include between 18 and 30 consecutive nucleotides of a coding region encoding a Bnip3Δex3 polypeptide (such as SEQ ID NO:2 or SEQ ID NO:6), an exon 3 from a coding region encoding a Bnip polypeptide (such as SEQ ID NO:10 or SEQ ID NO:11), or a complement thereof In one aspect, a polynucleotide of the present invention may be referred to as a sense strand. A sense strand has between 18 and 30 nucleotides, for instance, 18, 19, 20, 21, 22, 23, 24, 25, 26, 27, 28, 29, or 30 nucleotides. A sense strand is substantially identical, preferably, identical, to a target mRNA. As used herein, the term “identical” means the nucleotide sequence of the sense strand has the same nucleotide sequence as a portion of a polynucleotide, such as a target mRNA. As used herein, the term “substantially identical” means the sequence of the sense strand differs from the sequence of a target mRNA at 1, 2, 3, 4, 5, 6, 7, 8, 9 or 10 nucleotides, and the remaining nucleotides are identical to the sequence of a polynucleotide, such as a mRNA.

In one aspect, a polynucleotide of the present invention may be referred to as an antisense strand. The antisense strand may be between 18 and 30 nucleotides, for instance, 18, 19, 20, 21, 22, 23, 24, 25, 26, 27, 28, 29, or 30 nucleotides. An antisense strand is substantially complementary, preferably, complementary, to a target mRNA. The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide. An antisense strand that is “complementary” to another polynucleotide, such as a target mRNA, means the nucleotides of the antisense strand are complementary to a nucleotide sequence of a polynucleotide, such as a target mRNA. As used herein, the term “substantially complementary” means the antisense strand includes 1, 2, 3, 4, 5, 6, 7, 8, 9 or 10 nucleotides that are not complementary to a nucleotide sequence of a polynucleotide, such as a target mRNA.

The polynucleotides of the present invention also include a double stranded RNA (dsRNA) that includes a sense strand and antisense strand. In one embodiment the two strands of a dsRNA are complementary, and in another embodiment the two strands of a dsRNA are substantially complementary. Polynucleotides of the present invention also include the double stranded DNA polynucleotides that correspond to the dsRNA polynucleotides described herein. In one embodiment, the sense strand and the antisense strand of a double stranded polynucleotide have different lengths. Also included in the present invention are the single stranded RNA polynucleotides and single stranded DNA polynucleotides corresponding to the sense strands and antisense strands disclosed herein. It should be understood that the sequences disclosed herein as DNA sequences can be converted from a DNA sequence to an RNA sequence by replacing each thymidine nucleotide with a uracil nucleotide.

A polynucleotide of the present invention may include overhangs on one or both strands of a double stranded polynucleotide. An overhang is one or more nucleotides present in one strand of a double stranded polynucleotide that are unpaired, i.e., they do not have a corresponding complementary nucleotide in the other strand of the double stranded polynucleotide. An overhang may be at the 3′ end of a sense strand, an antisense strand, or both sense and antisense strands. An overhang is typically 1, 2, or 3 nucleotides in length. In one embodiment, the overhang is at the 3′ terminus and has the sequence thymine-thymine (or uracil-uracil if it is an RNA). Without intending to be limiting, such an overhang may be used to increase the stability of a dsRNA. If an overhang is present, it is preferably not considered a when determining whether a sense strand is identical or substantially identical to a target mRNA, and it is preferably not considered a when determining whether an antisense strand is complementary or substantially complementary to a target mRNA.

The sense and antisense strands of a double stranded polynucleotide of the present invention may also be covalently attached, for instance, by a spacer made up of nucleotides. Such a polynucleotide is often referred to in the art as a short hairpin RNA (shRNA). Upon base pairing of the sense and antisense strands, the spacer region typically forms a loop. The number of nucleotides making up the loop can vary, and loops between 3 and 23 nucleotides have been reported (Sui et al., Proc. Nat'l. Acad. Sci. USA, 99:5515-5520 (2002), and Jacque et al., Nature, 418:435-438 (2002)). In one embodiment, an shRNA includes a sense strand followed by a nucleotide loop and the analogous antisense strand. In one embodiment, the antisense strand can precede the nucleotide loop structure and the sense strand can follow.

Polynucleotides described herein may be modified. Such modifications can be useful to increase stability of the polynucleotide in certain environments. Modifications can include a nucleic acid sugar, base, or backbone, or any combination thereof. The modifications can be synthetic, naturally occurring, or non-naturally occurring. A polynucleotide of the present invention can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide-nucleic acids. Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-O-trifluoromethyl nucleotides, 2′-O-ethyl-trifluoromethoxy nucleotides, 2′-O-difluoromethoxy-ethoxy nucleotides, or 2′-deoxy nucleotides. Polynucletotides can be obtained commercially synthesized to include such modifications (for instance, Dharmacon Inc., Lafayette, Colo.).

Polynucleotides described herein may be biologically active. In one embodiment, a biologically active polynucleotide causes the post-transcriptional inhibition of expression, also referred to as silencing, of a target gene. The polynucleotides described herein may be referred to as RNAi, siRNA, shRNA, miRNA, or antisense oligonucleotides. Without intending to be limited by theory, after introduction into a cell a polynucleotide of the present invention will hybridize with a target mRNA if present and signal cellular polypeptides to cleave the target mRNA or to inhibit translation of the target mRNA. The result is the inhibition of expression of the polypeptide encoded by the mRNA. Whether the expression of a target gene is inhibited can be determined, for instance, by measuring a decrease in the amount of the target mRNA in the cell, measuring a decrease in the amount of polypeptide encoded by the mRNA, or by measuring a decrease in the activity of the polypeptide encoded by the mRNA.

An example of a target gene is the gene encoding Bnip3 and Bnip3Δex3. In one embodiment, a polynucleotide of the present invention includes between 18 and 30 nucleotides, for instance, 18, 19, 20, 21, 22, 23, 24, 25, 26, 27, 28, 29, or 30 nucleotides, and is complementary to nucleotides of a target Bnip3Δex3 mRNA. An example of a Bnip3Δex3 mRNA includes the nucleotides of SEQ ID NO:1. In one embodiment, the nucleotide sequence of such a polynucleotide is selected from nucleotides that include the junction of exons 2 and 4, for instance, nucleotides 197 and 198 of SEQ ID NO:1, or nucleotides 179 and 180 of SEQ ID NO:6. In one embodiment, the nucleotide sequence of such a polynucleotide is selected from nucleotides that encode the unique C-terminal region of Bnip3Δex3, e.g., LRKMILKEGKKLKAS (SEQ ID NO:3). An example of a nucleotide sequence encoding SEQ ID NO:3 is CTGAGGAAGATGATATTGAAAGAAGGAAAGAAGTTGAAAGCATCT (SEQ ID NO:16) which is nucleotides 198-243 of SEQ ID NO:1. In one embodiment, the nucleotide sequence of such a polynucleotide is selected from nucleotides that encode the unique C-terminal region of Bnip3Δex3, e.g., LRKIILREEEKLKVS (SEQ ID NO:8). An example of a nucleotide sequence encoding SEQ ID NO:6 is CTGAGGAAGATTATATTGAGAGAAGAAGAGAAGTTGAAAGTATCCTGA (SEQ ID NO:17) which is nucleotides 180-228 of SEQ ID NO:6.

In one embodiment, a polynucleotide of the present invention includes between 18 and 30 nucleotides, for instance, 18, 19, 20, 21, 22, 23, 24, 25, 26, 27, 28, 29, or 30 nucleotides, and is complementary to nucleotides of a target Bnip3 mRNA. In one embodiment, polynucleotides useful to target a Bnip3 mRNA include nucleotides corresponding to exon1, exon2, exon3, exon4, exons, exon6, or a combination thereof. In another embodiment, an example of polynucleotides useful to target a Bnip3 mRNA includes nucleotides corresponding to exon3. Targeting nucleotides of exon3 permits specific targeting of Bnip3 mRNA to decrease expression of a full length Bnip3 polypeptide while not targeting Bnip3Δex3 mRNA. An example of a nucleotide sequence corresponding to exon3 of a Bnip3 mRNA includes the nucleotides of SEQ ID NO:10 (an example of an exon3 from a human Bnip3 mRNA) and the nucleotides of SEQ ID NO:11 (an example of an exon3 from a rat Bnip3 mRNA).

### Vectors

A polynucleotide of the present invention may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector may provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, and artificial chromosome vectors. Examples of viral vectors include, for instance, lambda phage vectors, P1 phage vectors, M13 phage vectors, adenoviral vectors, adeno-associated viral vectors, lentiviral vectors, retroviral vectors, and herpes virus vectors. In one embodiment, a vector is capable of replication in a microbial host, for instance, a prokaryotic bacterium, such as E. coli. In one embodiment, a vector is capable of replication in a eukaryotic host, for instance, an animal cell. Preferably the vector is a plasmid. In one embodiment, a polynucleotide of the present invention can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. In some aspects, suitable host cells for cloning or expressing the vectors herein include prokaryotic cells. Suitable prokaryotic cells include eubacteria, such as gram-negative microbes, for example, E. coli. In other aspects, suitable host cells for cloning or expressing the vectors herein include eukaryotic cells. Suitable eukaryotic cells include cultured cells, such as human, primate, and murine cells. Vectors may be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells.

Polynucleotides of the present invention, such as polynucleotides encoding a Bnip3 polypeptide or a Bnip3Δex3 polypeptide, may be obtained from eukaryotic cells, such as mammalian cells, preferably human cells. Polynucleotides of the present invention may be produced in vitro or in vivo. For instance, methods for in vitro synthesis include, but are not limited to, chemical synthesis with a conventional DNA/RNA synthesizer. Commercial suppliers of synthetic polynucleotides and reagents for such synthesis are well known. Likewise, polypeptides of the present invention may be obtained from microbes, or produced in vitro or in vivo.

An expression vector optionally includes regulatory sequences operably linked to the polynucleotide of the present invention. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

Vectors may include constitutive, inducible, and/or tissue specific promoters for expression of a polynucleotide of the present invention in a particular tissue or intracellular environment, examples of which are known to one of ordinary skill in the art. Constitutive mammalian promoters include, but are not limited to, polymerase promoters as well as the promoters for the following genes: hypoxanthine phosphoribosyl transferase (HPTR), adenosine deaminase, pyruvate kinase, and β-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art.

Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

Examples of tissue-specific promoters include, but are not limited to, cardiac tissue specific promoters such as promoters from the following coding regions: an α-myosin heavy chain coding region, e.g., a ventricular α-myosin heavy chain coding region, β-myosin heavy chain coding region, e.g., a ventricular β-myosin heavy chain coding region, myosin light chain 2v coding region, e.g., a ventricular myosin light chain 2 coding region, myosin light chain 2a coding region, e.g., a ventricular myosin light chain 2 coding region, cardiomyocyte-restricted cardiac ankyrin repeat protein (CARP) coding region, cardiac α-actin coding region, cardiac m2 muscarinic acetylcholine coding region, ANP coding region, BNP coding region, cardiac troponin C coding region, cardiac troponin I coding region, cardiac troponin T coding region, cardiac sarcoplasmic reticulum Ca-ATPase coding region, and skeletal α-actin coding region. Further, chamber-specific promoters or enhancers may also be employed, e.g., for atrial-specific expression, the quail slow myosin chain type 3 (MyHC3) or ANP promoter may be used. Examples of ventricular myocyte-specific promoters include a ventricular myosin light chain 2 promoter and a ventricular myosin heavy chain promoter. Another tissue-specific promoter includes the promoter for creatine kinase, which has been used to direct expression in muscle and cardiac tissue and immunoglobulin heavy or light chain promoters for expression in B cells.

Other tissue specific promoters include the human smooth muscle α-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, β-galactosidase α-2,6 sialylkansferase promoter, insulin-like growth factor binding protein (IGFBP-1) promoter, aldolase B promoter, human transferrin promoter, and collagen type I promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter. Exemplary tissue-specific expression elements for breast cells include, but are not limited to, the human alpha-lactalbumin promoter. Exemplary tissue-specific expression elements for the lung include, but are not limited to, the cystic fibrosis transmembrane conductance regulator (CFTR) gene promoter.

An expression vector may optionally include a ribosome binding site and a start site (e.g., the codon ATG or GTG) to initiate translation of the transcribed message to produce the polypeptide. It may also include a termination sequence to end translation. A termination sequence is typically a codon for which there exists no corresponding aminoacetyl-tRNA, thus ending polypeptide synthesis. The polynucleotide used to transform the host cell may optionally further include a transcription termination sequence.

A vector introduced into a host cell optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell.

The polynucleotides described herein that have post-transcriptional inhibition of expression can be designed using methods that are routine and known in the art. For instance, polynucleotides that inhibit the expression of a Bnip3 or Bnip3Δex3 polypeptide may be identified by the use of cell lines and/or primary cells. A candidate polynucleotide is the polynucleotide that is being tested to determine if it decreases expression of a Bnip3 or Bnip3Δex3 polypeptide. Other methods are known in the art and used routinely for designing and selecting candidate polynucleotides. Candidate polynucleotides are typically screened using publicly available algorithms (e.g., BLAST) to compare the candidate polynucleotide sequences with mRNA sequences. Those that are likely to form a duplex with an mRNA expressed by a non-target coding region are typically eliminated from further consideration. The remaining candidate polynucleotides may then be tested to determine if they inhibit expression of one of the polypeptides described herein.

In general, candidate polynucleotides are individually tested by introducing a candidate polynucleotide into a cell that expresses the appropriate polypeptide. The candidate polynucleotides may be prepared in vitro and then introduced into a cell. The candidate polynucleotides may also be prepared by introducing into a cell a construct that encodes the candidate polynucleotide. Such constructs are known in the art and include, for example, a vector encoding and expressing a sense strand and an antisense strand of a candidate polynucleotide, and RNA expression vectors that include the sequence encoding the sense strand and an antisense strand of a candidate polynucleotide flanked by operably linked regulatory sequences, such as an RNA polymerase III promoter and an RNA polymerase III terminator, that result in the production of an RNA polynucleotide.

A cell that can be used to evaluate a candidate polynucleotide may be a cell that expresses the appropriate polypeptide. A cell can be ex vivo or in vivo. As used herein, the term “ex vivo” refers to a cell that has been removed from the body of a subject. Ex vivo cells include, for instance, primary cells (e.g., cells that have recently been removed from a subject and are capable of limited growth in tissue culture medium), and cultured cells (e.g., cells that are capable of extended culture in tissue culture medium). As used herein, the term “in vivo” refers to a cell that is within the body of a subject. For instance, an in vivo cell may be a cell present in an organ or a tumor. A cell may be obtained from a subject by, for example, biopsy of human breast tissue.

Examples of readily available cells expressing a Bnip3 or Bnip3Δex3 polypeptide include cultured cells such as, but not limited to, cells having isolated from cancers including, but not limited to, pancreatic, breast, or colorectal cancers. Sources of other suitable cells include primary cells obtained from biopsy, such as cells present in a pancreatic cancer tumor, a breast cancer tumor, a colorectal cancer tumor, or lymph nodes draining tissues harboring such tumors. Other cultured cells include cardiac cells. Other cells can also be modified to express one of the polypeptides by introducing into a cell a vector having a polynucleotide encoding the polypeptide that is to be silenced.

Candidate polynucleotides may also be tested in animal models. The study of various cancers in animal models (for instance, mice and rats) is a commonly accepted practice for the study of cancers. For instance, the nude mouse model, where human tumor cells are injected into the animal, is commonly accepted as a general model useful for the study of a wide variety of cancers. Candidate polynucleotides can be used in this and other animal models to determine if a candidate polynucleotide decreases one or more symptoms and/or signs associated with disease.

Methods for introducing a candidate polynucleotide into a cell, including a vector encoding a candidate polynucleotide, are known in the art and routine. When the cells are ex vivo, such methods include, for instance, transfection with a delivery reagent, such as lipid or amine based reagents, including cationic liposomes or polymeric DNA-binding cations (such as poly-L-lysine and polyethyleneimine) Alternatively, electroporation or viral transfection can be used to introduce a candidate polynucleotide, or a vector encoding a candidate polynucleotide. When the cells are in vivo, such methods include, but are not limited to, local or intravenous administration.

When evaluating whether a candidate polynucleotide functions to inhibit expression of one of the polypeptides described herein, the amount of target mRNA in a cell containing a candidate polynucleotide can be measured and compared to the same type of cell that does not contain the candidate polynucleotide. Methods for measuring mRNA levels in a cell are known in the art and routine. Such methods include quantitative reverse-transcriptase polymerase chain reaction (RT-PCR). Primers and specific conditions for amplification of an mRNA encoding a Bnip3 or Bnip3Δex3 polypeptide can be readily determined by the skilled person. An example of useful primers for RT-PCR includes 5′ ACCCACAGCTTTGGTGAGAA (SEQ ID NO:12) and 5′ CGCTTGTGTTTCTCATGATGCTG (SEQ ID NO:13) for Bnip3, and 5′CTGTGACAGTCTGAGGAA G (SEQ ID NO:14) and 5′ TGTTTCTCATGCTGAGAGT (SEQ ID NO:15) for Bnip3Δex3.

Other methods for evaluating whether a candidate polynucleotide functions to inhibit expression of one of the polypeptides described herein include monitoring the polypeptide. For instance, assays can be used to measure a decrease in the amount of polypeptide encoded by the mRNA, or to measure a decrease in the activity of the polypeptide encoded by the mRNA. Methods for measuring a decrease in the amount of a polypeptide include assaying for the polypeptide present in cells containing a candidate polynucleotide and comparing to the same type of cell that does not contain the candidate polynucleotide. Whether a cell expresses one of the polypeptides can be determined using methods that are routine and known in the art including, for instance, Western immunoblot, ELISA, immunoprecipitation, or immunohistochemistry. Western immunoblot and immunoprecipitation are generally used with ex vivo cells, and immunohistochemistry is generally used with in vivo cells.

A candidate polynucleotide that is able to decrease the expression of a Bnip3 or Bnip3Δex3 polypeptide by at least 20%, at least 30%, at least 40%, at least 50%, at least 60%, at least 70%, at least 80%, at least 90% or 100% when compared to a control cell, is considered to be a polynucleotide of the present invention.

The present invention also includes antibodies that specifically bind a polypeptide of the present invention. An antibody that specifically binds a Bnip3Δex3 polypeptide of the present invention, such as SEQ ID NO:2, SEQ ID NO:7, or a fragment thereof, does not bind to the Bnip3 polypeptide, such as SEQ ID NO:4 or SEQ ID NO:9.

Antibody may be produced using a polypeptide of the present invention, or a fragment thereof. The antibody may be polyclonal or monoclonal. Laboratory methods for producing, characterizing, and optionally isolating polyclonal and monoclonal antibodies are known in the art (see, for instance, Harlow E. et al., 1988, Antibodies: A laboratory manual, Cold Spring Harbor Laboratory Press, Cold Spring Harbor). For instance, a polypeptide of the present invention may be administered to an animal, such as a mammal or a chicken, in an amount effective to cause the production of antibody specific for the administered polypeptide. Optionally, a polypeptide may be mixed with an adjuvant, for instance Freund's incomplete adjuvant, to stimulate the production of antibodies upon administration. Production of antibody that specifically binds SEQ ID NO:2 (a Bnip3Δex3 polypeptide) and does not specifically bind Bnip3 (for instance, Genbank accession number NP_{—}004043, SEQ ID NO:4) may be accomplished by use of one or more fragments from SEQ ID NO:2 that have reduced identity with a series of amino acids in Bnip3. In one embodiment, a fragment of SEQ ID NO:2 includes at least 6 contiguous amino acids selected from LRKMILKEGKKLKAS (SEQ ID NO:3), which corresponds to amino acids 66 to 81 of SEQ ID NO: 2. The antibodies that result after administration of SEQ ID NO:3 or a fragment thereof would be expected to include those specifically binding to SEQ ID NO:2 and not specifically binding to Bnip3. Such specific antibody may be selected and purified using routine methods. Monoclonal antibodies made using SEQ ID NO:3 or a fragment thereof would likewise be expected to specifically bind a Bnip3Δex3 polypeptide and not bind a Bnip3 polypeptide.

An antibody of the present invention may be produced by recombinant methods known in the art. An antibody of the present invention may be modified by recombinant means to increase efficacy of the antibody in mediating the desired function.

Antibody fragments include at least a portion of the variable region of an antibody that specifically binds to its target. Examples of antibody fragments include, for instance, scFv, Fab, F(ab′)₂, Fv, a single chain variable region, and the like. Fragments of intact molecules can be generated using methods well known in the art and include enzymatic digestion and recombinant means.

Whether an antibody of the present invention specifically binds to a polypeptide of the present invention may be determined using methods known in the art. In one embodiment, specificity may be determined by testing antibody binding to SEQ ID NO:2 and a Bnip3 polypeptide, such as the one having the amino acid sequence described at Genbank accession number NP_{—}004043 (SEQ ID NO:4). In one embodiment, specificity may be determined by testing antibody binding to SEQ ID NO:7 and a Bnip3 polypeptide, such as the one having the amino acid sequence described at Genbank accession number AF243515 (SEQ ID NO:9).

An antibody of the present invention may be coupled (also referred to as conjugated) to a detectable label, e.g., a molecule that is easily detected by various methods. Examples include, but are not limited to, radioactive elements; enzymes (such as horseradish peroxidase, alkaline phosphatase, and the like); fluorescent, phosphorescent, and chemiluminescent dyes; latex and magnetic particles; cofactors (such as biotin); dye crystallites, gold, silver, and selenium colloidal particles; metal chelates; coenzymes; electroactive groups; oligonucleotides, stable radicals, and others. Methods for conjugating a detectable label to antibody vary with the type of label, and such methods are known and routinely used by the person skilled in the art.

Also provided herein are other molecules that specifically bind a polypeptide of the present invention. Examples of such molecules include DNA and/or RNA aptamers. Methods for making such molecules are known to the skilled person and are routine.

The present invention is also directed to compositions including one or more polynucleotides or polypeptides described herein. Such compositions typically include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. Additional active compounds can also be incorporated into the compositions.

A composition may be prepared by methods well known in the art of pharmacy. In general, a composition can be formulated to be compatible with its intended route of administration. Administration may be systemic or local. In some aspects local administration may have advantages for site-specific, targeted disease management. Local therapies may provide high, clinically effective concentrations directly to the treatment site, with less likelihood of causing systemic side effects.

In one embodiment, an active compound can be targeted to, for example, cardiac tissue (e.g., heart muscle) such as the right or left atrium or the right or left ventricle, or brain tissue. In one embodiment, an active compound can be targeted to, for example, neoplastic tissue, such as pre-malignant (pre-cancerous) tissue or malignant (cancerous) tissue (e.g., tumors). In one embodiment, an active compound can be targeted to, for example, blood vessels. In one embodiment, an active compound can be targeted to, for example, stroke injury, such as brain tissue. In one embodiment, an active compound can be targeted to, for example, stem cells, such as bone marrow stem cells.

Examples of routes of administration include parenteral, e.g., intravenous, intradermal, subcutaneous, oral, transdermal (topical), and transmucosal administration. In one embodiment, administration may include use of a delivery tool, such as a syringe, for direct injection into a specific site (e.g., during surgery) or by catheter.

Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a polynucleotide or polypeptide described herein) in the required amount in an appropriate solvent with one or a combination of ingredients such as those enumerated above, as required, followed by filtered sterilization. Generally, dispersions are prepared by incorporating the active compound into a sterile vehicle, which contains a dispersion medium and other ingredients such as from those enumerated above. In the case of sterile powders for the preparation of sterile injectable solutions, methods of preparation that may be used include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. An example of transdermal administration includes iontophoretic delivery to the dermis or to other relevant tissues.

The active compounds can also be administered by any method suitable for administration of polynucleotide agents, e.g., using gene guns, bio-injectors, and skin patches as well as needle-free methods such as the micro-particle DNA vaccine technology disclosed by Johnston et al. (U.S. Pat. No. 6,194,389). Additionally, intranasal delivery is possible, as described in, for instance, Hamajima et al. Clin. Immunol. Immunopathol., 88, 205-210 (1998). Delivery reagents such as lipids, cationic lipids, phospholipids, liposomes, and microencapsulation may also be used.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art.

In one embodiment, an active compound may be associated with a targeting group. As used herein, a “targeting group” refers to a chemical species that interacts, either directly or indirectly, with the surface of a cell, for instance with a molecule present on the surface of a cell, e.g., a receptor. The interaction can be, for instance, an ionic bond, a hydrogen bond, a Van der Waals force, or a combination thereof. Examples of targeting groups include, for instance, saccharides, polypeptides (including hormones), polynucleotides, fatty acids, and catecholamines. Another example of a targeting group is an antibody. The interaction between the targeting group and a molecule present on the surface of a cell, e.g., a receptor, may result in the uptake of the targeting group and associated active compound.

When a polynucleotide is introduced into cells using any suitable technique, the polynucleotide may be delivered into the cells by, for example, transfection or transduction procedures. Transfection and transduction refer to the acquisition by a cell of new genetic material by incorporation of added polynucleotides. Transfection can occur by physical or chemical methods. Many transfection techniques are known to those of ordinary skill in the art including, without limitation, calcium phosphate DNA co-precipitation, DEAE-dextrin DNA transfection, electroporation, naked plasmid adsorption, cationic liposome-mediated transfection (commonly known as lipofection),. Transduction refers to the process of transferring nucleic acid into a cell using a DNA or RNA virus.

A polynucleotide described herein may be used in combination with other agents assisting the cellular uptake of polynucleotides, or assisting the release of poylnucleotides from endosomes or intracellular compartments into the cytoplasm or cell nuclei by, for instance, conjugation of those to the polynucleotide. The agents may be, but are not limited to, peptides, especially cell penetrating peptides, protein transduction domains, and/or dsRNA-binding domains which enhance the cellular uptake of polynucleotides (Dowdy et al., US Published Patent Application 2009/0093026, Eguchi et al., 2009, Nature Biotechnology 27:567-571, Lindsay et al., 2002, Curr. Opin. Pharmacol., 2:587-594, Wadia and Dowdy, 2002, Curr. Opin. Biotechnol. 13:52-56. Gait, 2003, Cell. Mol. Life Sci., 60:1-10). The conjugations can be performed at an internal position at the oligonucleotide or at a terminal postions either the 5′-end or the 3′-end.

Toxicity and therapeutic efficacy of such active compounds can be determined by standard pharmaceutical procedures in cell cultures or experimental animals, e.g., for determining the ED₅₀ (the dose therapeutically effective in 50% of the population).

The data obtained from cell culture assays and animal studies can be used in formulating a range of dosage for use in humans. The dosage of such active compounds lies preferably within a range of circulating concentrations that include the ED₅₀ with little or no toxicity. The dosage may vary within this range depending upon the dosage form employed and the route of administration utilized. For a compound used in the methods of the invention, it may be possible to estimate the therapeutically effective dose initially from cell culture assays. A dose may be formulated in animal models to achieve a circulating plasma concentration range that includes the IC₅₀ (i.e., the concentration of the test compound which achieves a half-maximal inhibition of signs and/or symptoms) as determined in cell culture. Such information can be used to more accurately determine useful doses in humans.

The compositions can be administered one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of a polynucleotide or a polypeptide can include a single treatment or can include a series of treatments.

The inventors have found that alternative splicing of a Bnip3 gene provides a molecular switch regarding cell death by necrosis, apoptosis, autophagy, or a combination thereof, where the full length Bnip3 polypeptide and the splice variant Bnip3Δex3 polypeptide have distinct and opposing actions on cell survival. The full length Bnip3 polypeptide induces mitochondrial perturbations and cell death, while the splice variant Bnip3Δex3 polypeptide inhibits mitochondrial perturbations and cell death. Without intending to be limited by theory, the full length Bnip3 polypeptide targets the mitochondria while the splice variant Bnip3Δex3 polypeptide targets the endoplasmic reticulum. Moreover, it appears that the splice variant Bnip3Δex3 polypeptide will suppress the activity of the Bnip3 polypeptide.

The present invention includes methods of using the compositions described herein. In some embodiments, a method of the present invention include treating one or more symptoms or clinical signs of certain diseases in a subject. The subject is a mammal, including a member of the family Muridae (a murine animal such as rat or mouse), a primate, (e.g., monkey, human), a rabbit, a sheep, a goat, a dog, a pig, or a horse, preferably a human. As used herein, the term “disease” refers to any deviation from or interruption of the normal structure or function of a part, organ, or system, or combination thereof, of a subject that is manifested by a characteristic symptom or clinical sign. Diseases include, but are not limited to, cancers, such as pancreatic cancer, colorectal cancer, brain cancer, bladder cancer, and liver cancer. Such cancers are typically primary cancers, and can include cancerous cells that are not metastatic, and cancerous cells that are metastatic. A cancer may also include a metastasis, such as metastasis of a primary cancer. A metastatic cancer can be located in, for instance, the lymph nodes draining tissues containing a primary tumor. Other diseases include pathological conditions such as ischemia, hypoxia, damage to tissues resulting from exposure to biological agents, physical damage, and/or noxious chemicals. Examples of hypoxia include generalized hypoxia and tissue hypoxia, such as tissue hypoxia resulting from thrombosis. Examples of tissues that can be subject to hypoxia include, but are not limited to, cardiac tissues and brain tissues. Other diseases include, but are not limited to, acute myocardial infarction, myocardial ischemia, myocardial infarction, stroke, vascular disease, cardiac diseases involving ischemic, hypoxic hypertrophic cardiomyopathy, heart failure, and congenital birth defects of the blood vessel and heart muscle.

As used herein, the term “symptom” refers to subjective evidence of disease experienced by the patient and caused by disease. As used herein, the term “clinical sign,” or simply “sign,” refers to objective evidence of a disease present in a subject. Symptoms and/or signs associated with diseases referred to herein and the evaluation of such signs are routine and known in the art. Examples of signs of disease vary depending upon the disease. For instance, signs of cancer may include tumorigenesis, metastasis, and angiogenesis. Typically, whether a subject has a disease, and whether a subject is responding to treatment, may be determined by evaluation of signs associated with the disease.

Treatment of a disease can be prophylactic or, alternatively, can be initiated after the development of a disease. Treatment that is prophylactic, for instance, initiated before a subject manifests signs of a disease, is referred to herein as treatment of a subject that is “at risk” of developing a disease. An example of a subject that is at risk of developing a disease is a person having a risk factor. Risk factors include genetic markers. Treatment can be performed before, during, or after the occurrence of the diseases described herein. Treatment initiated after the development of a disease may result in decreasing the severity of the signs of the disease, or completely removing the signs.

In some embodiments described herein, a method includes administering to a subject an effect amount of a composition. The administering is under conditions suitable for introduction of an active compound, such as a polypeptide or a polynucleotide described herein, into a cell. Conditions that are “suitable” for an event to occur, such as introduction of a polypeptide or polynucleotide into a cell, or “suitable” conditions are conditions that do not prevent such events from occurring. Thus, these conditions permit, enhance, facilitate, and/or are conducive to the event. As used herein, an “effective amount” relates to a sufficient amount of an active compound to provide the desired effect. For instance, in one embodiment an “effective amount” is an amount effective to alleviate one or more signs and/or symptoms of disease. In some embodiments, an effective amount is an amount that is sufficient to effect a reduction in a symptom and/or sign associated with a disease. A reduction in a symptom and/or a sign is, for instance, a reduction of at least 10%, at least 20%, at least 30%, at least 40%, at least 50%, at least 60%, at least 70%, at least 80%, at least 90%, or at least 100% in a measured sign as compared to a control, a non-treated subject, or the subject prior to administration of the active compound. It will be understood, however, that the total daily usage of the compositions and formulations as disclosed herein will be decided by the attending physician within the scope of sound medical judgment. The exact amount required will vary depending on factors such as the type of disease being treated.

In one embodiment, a method of the present invention includes altering apoptosis, necrosis, autophagy, or the combination thereof, of a cell. The cell may be in vivo or ex vivo, and may be present in a tissue or an organ. For instance, the cell may be an ex vivo cancer cell or an ex vivo cardiac cell. The method may include expressing in a cell an effective amount of a polypeptide having Bnip3 antagonist activity wherein apoptosis, necrosis, autophagy, or the combination thereof, is altered in the cell compared to a control cell. In one embodiment, expressing the polypeptide in a cell may include introducing the polypeptide into the cell, and in another embodiment expressing the polypeptide in a cell may include introducing into the cell a polynucleotide encoding the polypeptide. In one embodiment, the method may include introducing into a cell a biologically active polynucleotide causing the post-transcriptional inhibition of expression of a Bnip3 polypeptide, wherein apoptosis, necrosis, autophagy, or the combination thereof, is altered in the cell compared to a control cell.

In one embodiment, a method of the present invention includes increasing the amount or activity of Bnip3Δex3 polypeptide in a cell. The cell may be in vivo or ex vivo, and may be present in a tissue or an organ. In one embodiment, increasing the amount of Bnip3Δex3 polypeptide in a cell may be used to treat certain diseases in which premature cell death occurs. Such diseases include, but are not limited to, acute myocardial infarction, hypoxia, myocardial ischemia, myocardial infarction, stroke, or vascular disease. The cell death may be due to apoptosis, necrosis, autophagy, or a combination thereof Thus, in one embodiment, a method of the present invention includes a method for altering apoptosis, preferably, decreasing apoptosis, altering necrosis, preferably decreasing necrosis, altering autophagy, preferably decreasing autophagy, or a combination thereof

A method for increasing the amount or activity of Bnip3Δex3 polypeptide in a cell includes introducing into a cell an effective amount of a composition, such as administering to a subject in need thereof an effective amount of a composition. The composition may include an active compound such as a Bnip3Δex3 polypeptide, a polynucleotide encoding a Bnip3Dex3 polypeptide, or an agent that increases the activity of a Bnip3Δex3 polypeptide. For instance, the composition may include a viral vector that encodes a Bnip3Δex3 polypeptide. In one embodiment, an active compound may be targeted to an appropriate cell.

In one embodiment, a method of the present invention includes decreasing the amount or activity of Bnip3 polypeptide in a cell. This results in an increase in the ratio of Bnip3Δex3 polypeptide:Bnip3 polypeptide in a cell. The cell may be in vivo or ex vivo, and may be present in a tissue or an organ. In one embodiment, increasing the ratio of Bnip3Δex3 polypeptide:Bnip3 polypeptide in a cell may be used to treat certain diseases in which premature cell death occurs. Such diseases include, but are not limited to, acute myocardial infarction, hypoxia, myocardial ischemia, myocardial infarction, stroke, or vascular disease. The cell death may be due to apoptosis, necrosis, autophagy, or a combination thereof. Thus, in one embodiment, a method of the present invention includes a method for altering apoptosis, preferably, decreasing apoptosis, altering necrosis, preferably decreasing necrosis, altering autophagy, preferably, decreasing autophagy, or a combination thereof.

A method for increasing the ratio of Bnip3Δex3 polypeptide:Bnip3 polypeptide in a cell includes introducing into a cell an effective amount of a composition, such as administering to a subject in need thereof an effective amount of a composition. The composition may include an active compound such as a Bnip3Δex3 polypeptide, a polynucleotide encoding a Bnip3Dex3 polypeptide, or a vector that encodes a biologically active polynucleotide causing the post-transcriptional inhibition of expression of a Bnip3 polypeptide. In one embodiment, an active compound may be targeted to an appropriate cell.

In one embodiment, a method of the present invention includes decreasing the amount or activity of Bnip3Δex3 polypeptide in a cell. The cell may be in vivo or ex vivo, and may be present in a tissue or an organ. In one embodiment, decreasing the amount of Bnip3Δex3 polypeptide in a cell may be used to treat certain diseases in which increasing the frequency of death of cells in tissue, such as diseased tissue, is desirable. Such diseases include, but are not limited to, cancer, including primary cancer and metastatic cancer. Cancer cells display uncontrolled cell growth, and the methods herein may lead to cell death by apoptosis, necrosis, autophagy, or a combination thereof. Thus, in one embodiment, a method of the present invention includes a method for altering apoptosis, preferably, increasing apoptosis, altering necrosis, preferably increasing necrosis, altering autophagy, preferably increasing autophagy, or a combination thereof.

A method for decreasing the amount or activity of Bnip3Δex3 polypeptide in a cell includes introducing into a cell an effective amount of a composition, such as administering to a subject in need thereof an effective amount of a composition. The composition may include an active compound such as an agent that decreases the activity of a Bnip3Δex3 polypeptide. For instance, the composition may include a vector that encodes a biologically active polynucleotide that causes the post-transcriptional inhibition of expression of a Bnip3Δex3 polypeptide. In one embodiment, an active compound may be targeted to an appropriate cell.

In one embodiment, the present invention provides a method for detecting a Bnip3Δex3 polypeptide. The method may include providing a cell, analyzing the cell for a polypeptide of the present invention, and determining whether the cell expresses the polypeptide.

In one embodiment, a method may include determining whether death of cells may be decreased or increased. A biological sample may be used and analyzed to determine whether cells present in the biological sample express a Bnip3Δex3 polypeptide. In one embodiment, the absence of Bnip3Δex3 polypeptide compared to a control cell indicates that death of the cells in the diseased tissue can be decreased. In one embodiment, the presence of Bnip3Δex3 polypeptide indicates that death of the cells in the diseased tissue can be increased. As used herein, a “biological sample” refers to a sample of tissue or fluid isolated from a subject, including but not limited to, for example, blood, plasma, serum, urine, bone marrow, bile, spinal fluid, lymph tissue and lymph fluid, samples of the skin, external secretions of the skin, respiratory, intestinal, and genitourinary tracts, tears, saliva, blood cells, and organs such as heart and brain. Biological samples can also include sections of tissues such as biopsy samples, and frozen sections taken for histologic purposes. Biological samples also include explants and primary and/or transformed cell cultures derived from patient tissues. A biological sample can be provided by removing a tissue sample or a sample of cells from a subject, but can also be accomplished by using previously isolated cells (e.g., isolated by another person, at another time, and/or for another purpose). Archival tissues, such as those having treatment or outcome history can also be used.

In one embodiment, an antibody that specifically binds a Bnip3Δex3polypeptide, for instance, an antibody that specifically binds SEQ ID NO:3 or SEQ ID NO:8, may be used. In one embodiment, the presence or absence of a Bnip3Δex3 polypeptide may be inferred from the amount of mRNA encoding a Bnip3Δex3 polypeptide. A method for measuring mRNA includes polymerase chain reaction (PCR) based methods, and such methods are routine and known to the skilled person.

The detection of a Bnip3Δex3 polypeptide may be used for diagnostic and prognostic applications. Such a method may be used to evaluate treatment options for a subject. For instance, when a subject has a disease that may be treated by altering the amount or activity of Bnip3Δex3 polypeptide in a cell, such a method may indicate that treatment to alter the amount or activity of a Bnip3Δex3 polypeptide is appropriate. In one embodiment, when a subject has a disease that may be treated by increasing the amount or activity of Bnip3Δex3 polypeptide in a cell (for instance, myocardial infarction), such a method may indicate that treatment to increase the amount or activity of a Bnip3Δex3 polypeptide is appropriate. In one embodiment, when a subject has a disease that may be treated by decreasing the amount or activity of Bnip3Δex3 polypeptide in a cell (for instance, a cancer), such a method may indicate that treatment to decrease the amount or activity of a Bnip3Δex3 polypeptide is appropriate.

The present invention also provides a method for targeting a polypeptide to the endoplasmic reticulum of a cell. Such a method may include providing a cell that includes a polypeptide having a C-terminal polypeptide as described herein. The C-terminal polypeptide may be a fusion that includes an additional amino acid sequence not normally or naturally associated with the polypeptide, e.g., the polypeptide sequence may be something other than the amino acid sequence encoding by exons 1-2 of a Bnip3 gene. The C-terminal polypeptide may be located at the N-terminal end of a fusion polypeptide, at the C-terminal end of a fusion polypeptide, or at some other location. The cell may already include a polynucleotide that encodes the fusion polypeptide, or the method may further include introducing into the cell a polynucleotide that encodes the fusion polypeptide. The method further includes incubating the cell under conditions suitable for the fusion polypeptide to migrate to the endoplasmic reticulum.

The present invention also provides a method for identifying a compound that increases the amount or promotes the activity of a Bnip3Δex3 polypeptide (e.g., increases the ability of Bnip3Δex3 polypeptides to inhibit the cytotoxic action of Bnip3), or decreases the amount or inhibits the activity of a Bnip3Δex3 polypeptide (e.g., decreases the ability of Bnip3Δex3 polypeptides to inhibit the cytotoxic action of Bnip3). The method may include combining a Bnip3Δex3 polypeptide with a compound, and determining whether the agent alters the activity of the polypeptide. A compound can be obtained using any of the numerous approaches in combinatorial library methods known in the art, including biological libraries and synthetic library methods. The sources for potential agents to be screened include also include, for instance, fermentation media of bacteria and fungi, and cell extracts of plants and other vegetations.

The present invention also provides kits for practicing the methods described herein. A kit includes one or more of the polynucleotides and/or polypeptides described herein in a suitable packaging material in an amount sufficient for at least one use. Optionally, other reagents such as buffers and solutions needed to practice the invention are also included. Instructions for use of the packaged polynucleotides and/or polypeptides are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the polynucleotides and/or polypeptides can be used for the methods described herein. In addition, the packaging material contains instructions indicating how the materials within the kit are employed to practice the methods. As used herein, the term “package” refers to a solid matrix or material such as glass, plastic, paper, foil, and the like, capable of holding within fixed limits the polynucleotides and/or polypeptides. Thus, for example, a package can be a glass vial used to contain appropriate quantities of the polynucleotides and/or polypeptides. “Instructions for use” typically include a tangible expression describing the conditions for use of the polynucleotides and/or polypeptides.

The present invention is illustrated by the following examples. It is to be understood that the particular examples, materials, amounts, and procedures are to be interpreted broadly in accordance with the scope and spirit of the invention as set forth herein.

### EXAMPLE 1

This example describes the identification of a novel previously unrecognized spliced variant of Bnip3 (Bnip3Δex3) generated by alternative splicing of exon3 exclusively in cardiac myocytes subjected to hypoxia. Sequencing of Bnip3Δex3 revealed a frame shift mutation that terminated transcription up-stream of exon5 and exon6 ablating translation of the critical carboxyl terminal transmembrane domain crucial for mitochondrial localization and cell death. Notably, while the 26kDa Bnip3 protein (Bnip3FL) encoded by full length mRNA was localized to mitochondria and provoked cell death the 8.2kDa Bnip3Δex3 protein encoded by the truncated spliced mRNA was defective for mitochondrial targeting but interacted with Bnip3FL resulting in less association of Bnip3FL with mitochondria and diminished apoptotic and necrotic cell death. Forced expression of Bnip3FL in cardiac myocytes or Bnip3−/− mouse embryonic fibroblasts triggered widespread cell death that was inhibited by co-expression of Bnip3Δex3. Conversely, RNA interference targeted against sequences encompassing the unique exon2-exon4 junction of the Bnip3Δex3 sensitized cardiac myocytes to mitochondrial perturbations and cell death induced by Bnip3FL. Given the otherwise lethal consequences of de-regulated Bnip3FL expression in post-mitotic cells, these findings reveal a novel intrinsic defense mechanism that opposes the mitochondrial defects and cell death of ventricular myocytes that is obligatorily linked and mutually dependent upon alternative splicing of Bnip3FL during hypoxia or ischemic stress. Thus, therapeutic interventions designed to selectively promote Bnip3Δex3 activity in the heart may prove beneficial in suppressing cell death during hypoxia.

### Materials and Methods

### Cell Culture

Post-natal ventricular myocytes from 1-2 day old Sprague-Dawley rats were subjected to hypoxia for 24 hr in an air-tight chamber under serum free culture conditions continually gassed with 95% N2, 5% CO2 , PO2<5 mmHg, as previously reported (Regula et al., 2002, Circ. Res., 91:226-231, Baetz et al., 2005, Circulation, 112:3777-3785, Gurevich et al., 2001, Circulation, 103:1984-1991). Bnip3−/− MEFS were cultured as previously reported (Diwan et al., 2007, J. Clin. Invest., 117:2825-2833).

Quantitative Real Time RT-PCR (qRT-PCR) and Radioactive Semi-Quantitative RT-PCR

Total RNA (1 μg) from post-natal cardiac myocytes or adult heart tissue was reverse transcribed with oligo dT20 (Invitrogen), and one-twentieth of the reaction was then amplified with gene specific primers for Bnip3, Bnip3Δex3 or house keeping control gene L32, respectively. Real time RT-PCR was performed using iQ5 multicolor Real-time PCR detection system (BioRad). For radioactive RT-PCR, 1 μg of the total RNA from post-natal cardiac myocytes was reverse transcribed with oligo dT(18), and one-tenth of the reaction was then amplified in 25 cycles of PCR with Bnip3 primers, and the reverse primer was ³²P-labeled. The PCR products were resolved on 8% polyacrylamide/8 M urea denaturing gels. The gel was dried, exposed, and scanned in a Phosphorlmager (Fuji Medical Systems, Roselle, Ill., United States).

### Cloning and Constructs

Bnip3 gene was amplified from rat genomic DNA while Bnip3Δex3 PCR product was purified from radioactive gel. The primers 5′ ACCCACAGCTTTGGTGAGAA (SEQ ID NO:18) and 5′ CGCTTGTGTTTCTCATGATGCTG (SEQ ID NO:19) were used to amplify Bnip3, and 5′CTGTGACAGTCTGAGGAA G (SEQ ID NO:20) and 5′ TGTTTCTCATGCTGAGAGT (SEQ ID NO:21) were used to amplify Bnip3Δex3. Both PCR products were cloned into pcDNA4/HisMax TOPO TA expression vector or pcDNA6.2/EmGFP vector (Invitrogen) to generate expression plasmids encoding either Bnip3 or Bnip3Δex3 His-tag or GFP-fusion constructs. Bnip3 and Bnip3Δex3 expression adenovirus were constructed by cloning PCR products of Bnip3 or Bnip3Δex3 into pAd/CMVN5-DEST vector (Invitrogen). Sh-RNA-Bnip3FL was designed to directly target Bnip3 exon3 to knock down full length Bnip3. Sh-RNA-Bnip3FL was constructed by cloning a specific double-stranded oligonucleotide into adenovirus expression vector pBLOCK-iT 6-DEST purchased from Invitrogen. The double-stranded oligonucleotide was generated by annealing the sense and anti-sense oligonucleotides with specific sequences to target Bnip3 exon3. The Sh-RNA-Bnip3FL included nucleotides CACCGACACAAGATACCAACAGCGAACTGTTGGTATCTTGTGGTGTC (SEQ ID NO:22). Sh-RNA-Bnip3Δex3 directed against the exon2-exon4 junction sequence was also constructed. The Sh-RNA-Bnip3Δex3 included nucleotides CACUGUGACAGUCUGAGGAUU (SEQ ID NO:23). Bnip3Δex3 small interfering RNA (siRNA-Bnip3Δex3) was designed to target Bnip3 exon2-exon4 junction to selectively knock down Bnip3Δex3. The siRNA-Bnip3Δex3 oligonucleotide was purchased from Invitrogen, and its sequence was CACUGUGACAGUCUGAGGAUU (SEQ ID NO:24). All the constructs were confirmed by DNA sequencing.

### Cell Viability

Cell viability was determined using the vital dyes calceinacetoxymethylester (2 μM) to determine the number of living cells (green fluorescence) and ethidium homodimer-1 (2 μM) to determine the number of dead cells (red fluorescence), (Molecular Probes, Eugene Oreg.) as previously reported (Regula et al., 2002, Circ. Res., 91:226-231). Cells were analyzed from at least n=3-4 independent myocyte cultures counting >300 cells for each condition tested. Data are expressed as mean±S.E. percent reduction from control.

### Western Blot Analysis

Cardiac myocyte cell lysates (20 μg) were resolved on a 4-20% SDS-PAGE gel. Bnip3 proteins were detected using antibodies as previously reported (Regula et al., 2002, Crirc. Res., 91:226-231). Cytoplasmic S-100 and mitochondrial fractions of cardiac lysate were prepared as previously reported (Gurevich et al., 2001, Circulation. 103:1984-1991, Bialik et al., 1999, Circ. Res., 85:403-414). Murine antibodies directed toward VDAC 1 (1 μg/ml, Cell Signaling) were used to verify the integrity of the preparation (Moissac et al., 2000, J. Mol. Cell Cardiol., 32:53-63). Bound proteins were visualized using enhanced chemiluminesence reagents (Pharmacia Inc).

### Mitochondrial Permeability Transition ΔΨm and Reactive Oxygen Species (ROS)

To monitor mitochondrial membrane potential (ΔΨm), myocytes were incubated with 50 nM tetra-methylrhodamine methyl ester perchlorate (TMRM), (Molecular Probes, Eugene Oreg.) (Gurevich et al., 2001, Circulation. 103:1984-1991, Regula and Kirshenbaum, 2001, J. Mol. Cell Cardiol., 33:1435-1445). Cells were visualized using an Olympus AX-70 Research fluorescence microscope (Regula and Kirshenbaum 2001, J. Mol. Cell Cardiol., 33:1435-1445, Regula et al., 2002, Circ. Res., 91:226-231). To monitor ROS production, cells were incubated with 2.5 μM Dihydroethidium or 10 μM CM-H2DCFDA (5-(and-6)-chloromethyl-2′,7′-dichlorodihydrofluorescein diacetate, acetyl ester) (Molecular Probes, Eugene Oreg.) at 37° C. for 30 min. Cells were visualized by epifluorescence microscopy as described above.

### Flow Cytometry Analysis

Postnatal ventricular myocytes were stained with DAPI and APC-Annexin V (BD Biosciences) and analyzed by flow cytometry following a previously described protocol (Zhang et al., 2010, J. Immunol., 184:164-172).

### Statistical Analysis

Multiple comparisons between groups were determined by ANOVA. Unpaired two-tailed Student's t-test was used to compare mean differences between groups. Differences were considered to be statistically significant to a level of p<0.05. In all cases the data was obtained from at least n=3 to 4 independent myocyte isolations using n=3 replicates for each condition tested.

### Results

### Hypoxia-Induced Alternatively Spliced Variant Bnip3ΔEx3 in Ventricular Myocytes.

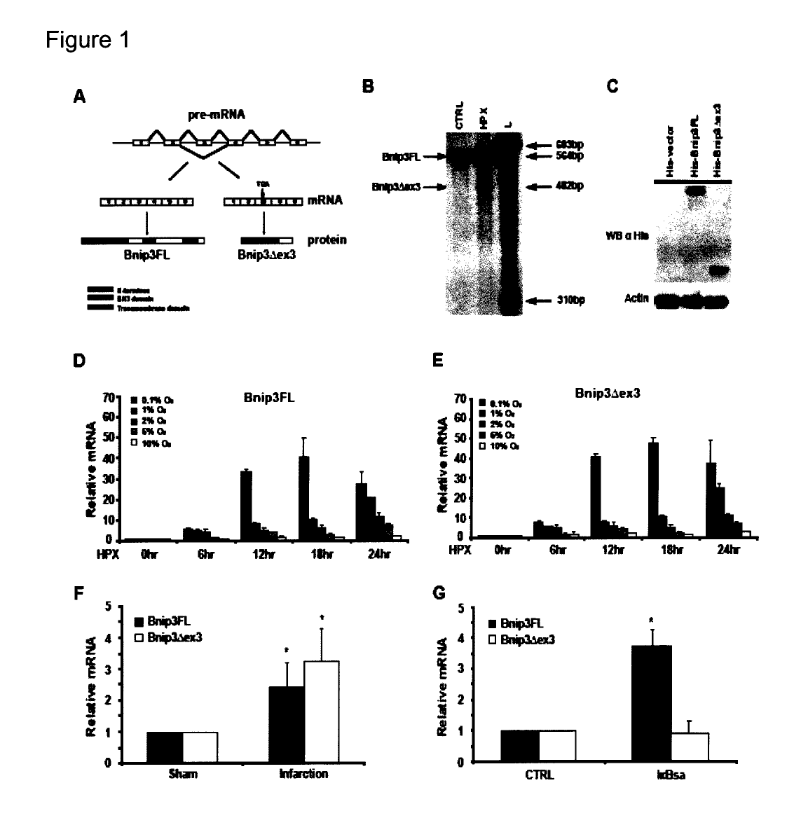

We previously established hypoxia-induced activation of the Bnip3 promoter, resulting in a Bnip3FL transcript of 564 base pairs. Sequence analysis of the PCR product verified the Bnip3FL mRNA included exons 1 through 6 and encoded for a Bnip3 protein of approximately 26 kDa. Interestingly, during the course of our studies we discovered a faster migrating mRNA transcript of 482 base pairs by radioactive PCR that was only detectable in ventricular myocytes subjected to hypoxia. Notably, baseline mRNA levels of this smaller mRNA were virtually undetectable relative to Bnip3FL in normoxic cells. Sequence analysis revealed the smaller mRNA band in hypoxia cells was an isoform of Bnip3FL that was missing 82 nucleotides encompassing exon3 (FIG. 1B). As shown in FIG. 1A, the constitutive Bnip3 mRNA isoform is includes exons 1 through 6 and encoded for a full length Bnip3 protein of 187 amino acids, and contains three functional domains: N-terminus, BH3 domain and transmembrane domain. The hypoxia induced alternatively spliced Bnip3Δex3 mRNA isoform was characterized by the skipping of exon3, which creates a premature stop codon in exon4. The Bnip3Δex3 mRNA is 482 base-pair and encodes a truncated protein of 76 amino acids which lacks the putative BH3 domain and carboxyl terminal transmembrane domain but retains the N-terminus of full length Bnip3. Further inspection of the spliced isoform revealed that the exclusion of exon3, and subsequent fusion of exon 2 and exon 4, introduced a premature stop codon terminating transcription up-stream of exon 5 and exon 6. Hence, the truncated Bnip3 protein encoded by the alternatively spliced Bnip3 mRNA that we designate Bnip3Δex3, has a predicted mass of 8.2 kDa, and retains the N-terminus of full length Bnip3, but lacks the putative BH3 and carboxyl-terminal transmembrane domain crucial for integrating into mitochondrial membranes (FIG. 1A-C).

As a first step toward determining the physiological significance of Bnip3Δex3 in ventricular myocytes, we assessed the relative temporal expression of Bnip3Δex3 isoform in ventricular myocytes under physiological conditions. For these studies, we designed qPCR primers that specifically amplified the Bnip3Δex3 isoform in cardiac cells under normoxic and hypoxic conditions. As shown in FIGS. 1D and E, our findings indicate that full-length Bnip3 and the Bnip3 splice variant are both induced in a manner dependent upon the degree and severity of hypoxia. We determined that moderate to severe hypoxia of 0.1%-5% not only activates Bnip3 gene transcription, but promotes Bnip3Δex3 splicing. This finding is in full agreement with our previous hypoxia studies demonstrating that a minimum threshold of oxygen deprivation is required to activate the Bnip3 promoter in ventricular myocytes (Regula et al., 2002, Circ. Res., 91:226-231).

To verify that this alternative splicing was not restricted to neonatal myocytes, we next tested whether Bnip3 alternative splicing occurs in adult myocytes under relevant physiological conditions in vivo. For these studies we assessed the presence of Bnip3FL and Bnip3Δex3 isoforms in adult myocardium following myocardial infarction in vivo. As shown in FIG. 1F, in contrast to sham operated control hearts, a marked increase in Bnip3FL and Bnip3Δex3 isoforms were detected in myocytes following myocardial infarction. These findings corroborate our in vitro data and verify that alternative splicing of Bnip3FL occurs in adult ventricular myoctyes under relevant ischemic stress conditions imposed by myocardial infarction. Since we previously established that the induction of Bnip3FL under hypoxia is due to the transcriptional derepression of the Bnip3 promoter by the displacement of NF-κB inhibitory complexes (Shaw et al., 2008, Proc. Natl. Acad. Sci. U.S.A., 105:20734-20739), we were prompted to test whether the Bnip3Δex3 splice variant is generated by a hypoxia-regulated mechanism or simply related to increased cellular processing of Bnip3 pre-mRNA transcript levels. Therefore, we next assessed whether Bnip3 pre-mRNA would undergo alternative splicing under basal conditions in the absence of hypoxic signal. To test this possibility, we rendered the ventricular myocytes defective for NF-κB signaling with a non-phosphorylatable mutant of IκBα that we had previously shown to de-repress the Bnip3 promoter and increase Bnip3FL gene transcription (Shaw et al., 2008, Proc. Natl. Acad. Sci. U.S.A., 105:20734-20739). As shown by real time PCR in FIG. 1G, endogenous full length Bnip3 mRNA was significantly increased in ventricular myocytes defective for NF-κB signaling—a finding consistent with our earlier work for the regulation of Bnip3 transcription by NF-κB. In contrast, we did not detect the alternative Bnip3Δex3 isoform in these cells, despite the increase in full length Bnip3 transcript levels. These findings confirm that alternative splicing of Bnip3 is a hypoxia regulated process. Bnip3ΔEx3 Suppresses ROS, Mitochondrial ΔΨm and Cell Death in Ventricular Myocytes

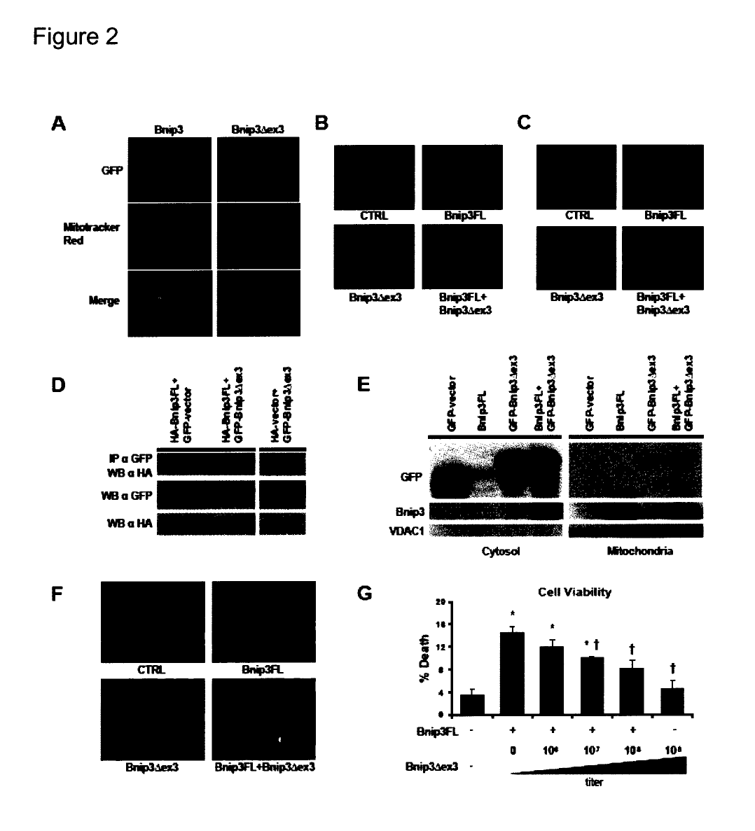

To assess the physiological significance of the Bnip3Δex3 in cardiac myocytes, we designed eukaryotic expression vectors encoding GFP-fusion proteins of Bnip3FL and Bnip3Δex3. As shown by epifluorescence microscopy in FIG. 2A, expression of Bnip3FL in cells was punctuate and co-localized with MitoTracker Red to mitochondrial membranes—consistent with our earlier work demonstrating that Bnip3FL associates with mitochondria. However, in contrast to Bnip3FL, expression of Bnip3Δex3 in cells was not localized to mitochondria. Cell fractionation experiments confirmed that Bnip3FL, but not Bnip3Δex3, was associated with mitochondria (FIG. 2E), verifying our epifluorescence data for the localization of Bnip3FL to mitochondria. Given early work establishing that localization of Bnip3FL to mitochondrial membranes is crucial for provoking permeability transition pore opening and cell death (Regula et al., 2002, Circ. Res., 91:226-231), we reasoned that an absence of Bnip3Δex3 localized at mitochondria may indicate its use as an intrinsic dominant-negative inhibitor of Bnip3FL to curtail mitochondrial injury and cell death during hypoxia.

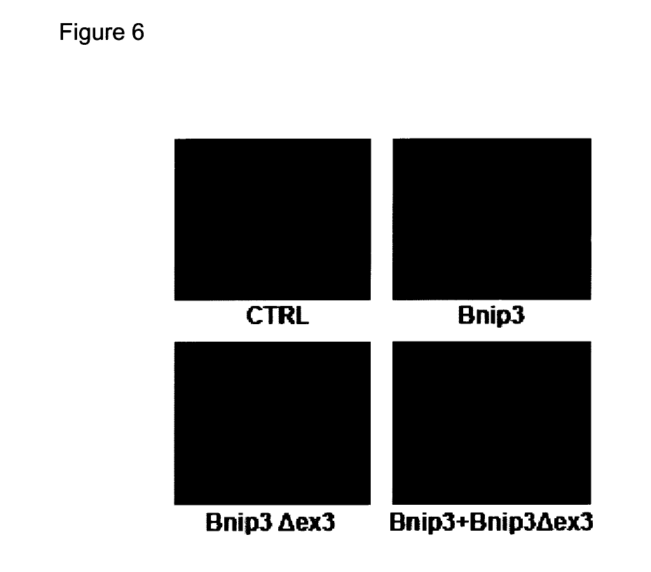

Therefore, to test this possibility we assessed whether Bnip3Δex3 influences mitochondrial ΔΨm changes induced by Bnip3FL. As shown in FIG. 2B, in contrast to control cells or cells expressing Bnip3Δex3, a marked reduction in mitochondrial TMRM staining was only observed in cells expressing Bnip3FL, a finding concordant with our epi-fluorescence data for localization of Bnip3FL to mitochondria. Moreover, a significant increase in reactive oxygen species (ROS) was observed in the presence of Bnip3FL, but not the Bnip3Δex3 (FIG. 2C and FIG. 6). Importantly, the loss of mitochondrial ΔΨm and ROS production induced by Bnip3FL in ventricular myocytes was suppressed by forced expression of Bnip3Δex3 (FIG. 2B-C). Since earlier work by our laboratory established mitochondrial membrane integration of Bnip3FL as critical for disrupting mitochondrial function, we reasoned that Bnip3Δex3 may interact with Bnip3FL preventing its ability to integrate into mitochondrial membranes. As shown by Western blot analysis in FIG. 2D, Bnip3Δex3 immunoprecipitated with Bnip3FL, indicating that Bnip3Δex3 forms protein interactions with Bnip3FL. Importantly, cell fractionation studies revealed that less Bnip3FL was associated with mitochondrial membranes in the presence of Bnip3Δex3, supporting the notion that Bnip3Δex3 suppresses mitochondrial perturbations by disrupting integration of Bnip3FL into mitochondrial membranes.

Based on these findings, we tested the role of Bnip3Δex3 on cell survival. For these studies, ventricular myocytes were infected with adenoviruses encoding either Bnip3FL or Bnip3Δex3 and stained with vital dyes calcein-AM and ethidium homodimer-1 to mark the number of living (green) and dead (red) cells, respectively. As shown in FIG. 2F, a significant increase in myocyte death was observed in the cells expressing Bnip3FL compared to vector control cells. Importantly, myocytes expressing Bnip3Δex3 were indistinguishable from vector control cells, with respect to cell viability, indicating that unlike Bnip3FL, the alternative spliced variant Bnip3Δex3 was not cytotoxic and did not provoke cell death. Furthermore, co-expression of Bnip3Δex3 rescued cell death induced by Bnip3FL in a dose dependent manner, to comparable levels of vector control cells (FIG. 2G). These findings support our notion that Bnip3Δex3 promotes survival by acting as an endogenous inhibitor of Bnip3FL.

### Bnip3ΔEx3 Abrogates Hypoxia-Induced Loss of Mitochondrial ΔΨm and Cell Death

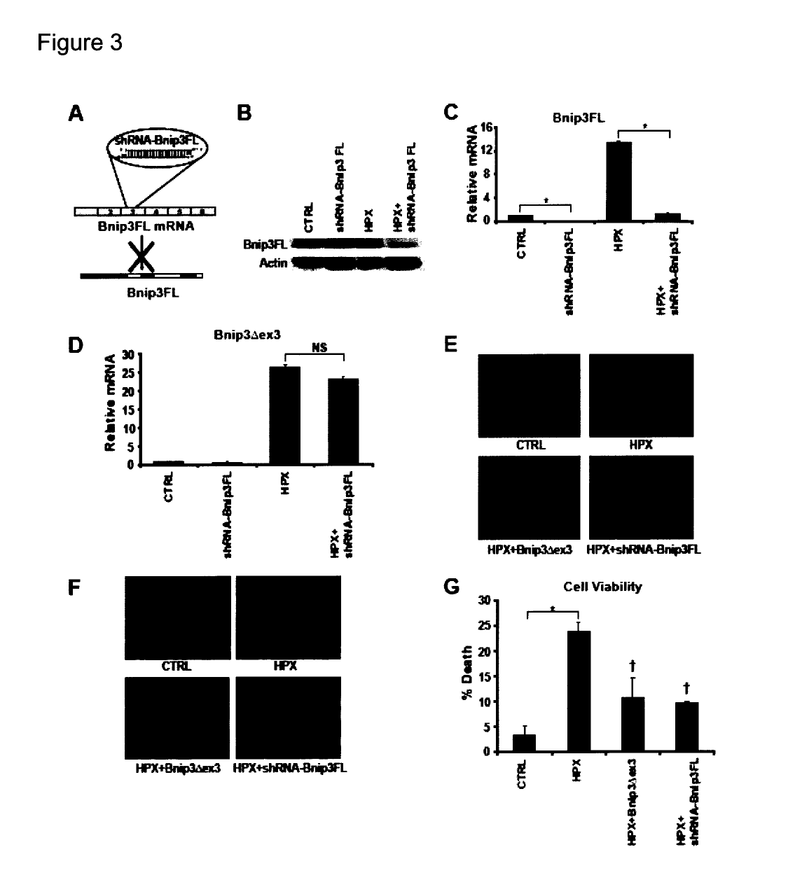

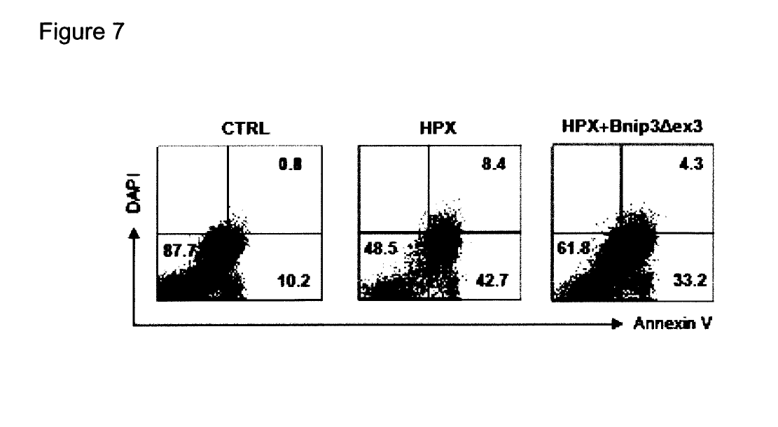

Collectively, our data strongly suggest that Bnip3Δex3 promotes cell survival in ventricular myocytes by opposing the actions of Bnip3FL under physiological conditions. To test this notion we utilized three independent, but complementary strategies, to assess the impact of Bnip3Δex3 on mitochondrial membrane potential (ΔΨm) and cell viability during hypoxia. First, we tested whether forced expression of Bnip3Δex3 would be sufficient to suppress hypoxia-induced loss of mitochondrial ΔΨm in ventricular myocytes. As shown in FIG. 3E, in contrast to normoxic control cells a significant reduction in &I′m was observed in ventricular myocytes during hypoxia—a finding concordant with our earlier work. Notably, the observed loss of mitochondrial ΔΨm during hypoxia was prevented in cells expressing Bnip3Δex3. Moreover, the preservation of mitochondrial ΔΨm by Bnip3Δex3 during hypoxia coincided with a marked reduction in cell death by apoptosis and necrosis and significantly increased cell viability compared to cells subjected to hypoxia alone (FIG. 3F-G, FIG. 7). Importantly, Bnip3Δex3 had no effect on the expression levels of Bcl-2 family members, Bax, Bak, or Beclin-1, excluding the possibility that Bnip3Δex3 promotes survival by influencing the expression levels of these factors.

To prove a survival role for endogenous Bnip3Δex3, we assessed whether selectively inhibiting full length Bnip3 isoform would influence mitochondrial function and cell viability during hypoxia. For these studies we designed a shRNA (shRNA-Bnip3FL) specifically targeting sequences against Bnip3 exon3 as a means to selectively knock-down Bnip3FL. We reasoned that since exon3 sequences are only present in the Bnip3FL isoform and not the Bnip3Δex3 isoform, endogenous Bnip3FL and not Bnip3Δex3 would be affected by the shRNA knock-down (FIG. 3A). As shown in FIG. 3B-C, in contrast to normoxic control cells a significant increase in Bnip3FL gene and protein expression was observed in cells subjected to hypoxia, which is in agreement with our earlier work involving hypoxia-induced transcription of Bnip3 (Regula et al., 2002, Circ. Res., 91:226-231).

In the presence of shRNA-Bnip3FL, however, expression of Bnip3FL was markedly inhibited, while Bnip3Δex3 expression was unaffected, in cells during hypoxia (shown in FIGS. 3B and 3D, respectively). Notably, during hypoxia mitochondrial membrane ΔΨm and cell viability were indistinguishable from normoxic control cells following Bnip3FL knock-down (FIGS. 3E and 3F). Importantly, our preliminary and published studies verified the specificity and integrity of the shRNA against Bnip3FL (Shaw et al., 2008, Proc. Natl. Acad. Sci. U.S.A., 105:20734-20739), thereby excluding the possibility for off target effects of the Bnip3FL RNA interference on cell survival. Together these findings strongly suggest that Bnip3Δex3 protects cardiac myocytes against mitochondrial defects and cell death induced by Bnip3FL during hypoxia.

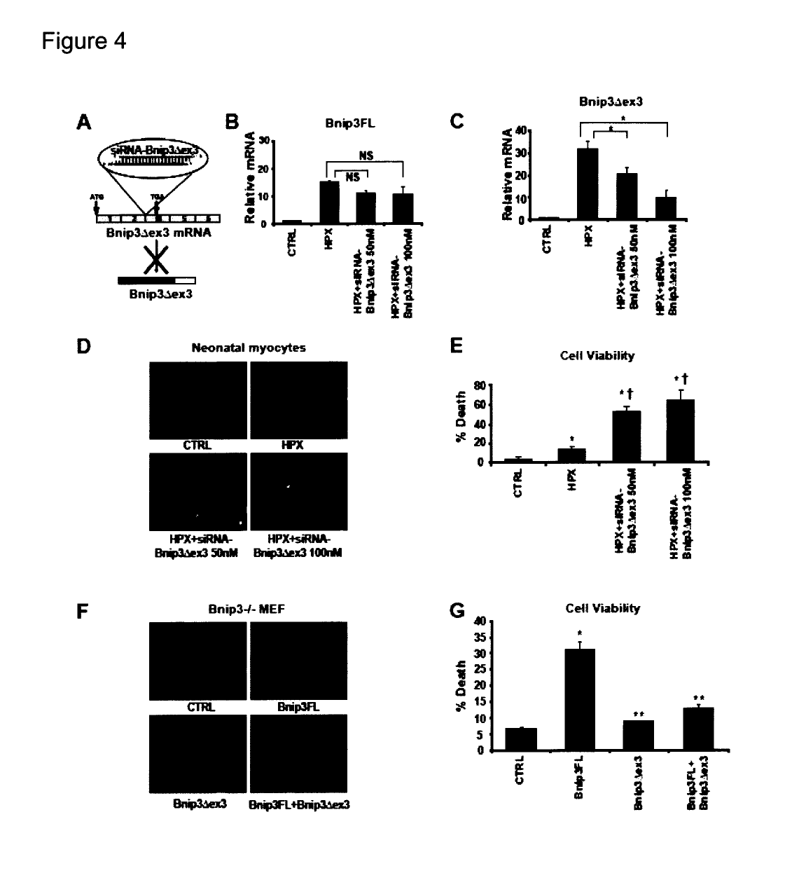

Secondly, to validate the notion that Bnip3Δex3 plays an important survival role in ventricular myocytes by antagonizing or opposing Bnip3FL, we reasoned that cells deficient or defective for Bnip3Δex3 isoform would be more susceptible to hypoxic injury. To test this possibility, we conducted reciprocal experiments in which we rendered ventricular myocytes defective for Bnip3Δex3 during hypoxia. For these studies, we designed siRNA targeted against the unique sequences encompassing the exon2-exon4 junction that is present only in the alternative spliced variant Bnip3Δex3 and not in the Bnip3FL FIG. 4 (panel A-C). Therefore, this would allow us to selectively knock-down Bnip3Δex3 isoform without influencing Bnip3FL. Remarkably, in contrast to control cells, viability was dramatically reduced in cells following Bnip3Δex3 knock-down during hypoxia, FIG. 4 (panels D-E). These findings are consistent with our interaction data for Bnip3Δex3 and Bnip3FL (FIG. 2D), and the ability of Bnip3Δex3 to suppress cell death by disrupting integration of Bnip3FL into mitochondria (FIG. 2E).

Thirdly, to further prove that Bnip3Δex3 opposes the cytotoxic actions of Bnip3FL to promote survival, we next tested the impact of Bnip3 isoforms on cell viability in Bnip3−/− mouse embryonic fibroblasts (MEFs). Because these cells are deficient for generating both Bnip3 isoforms, we reasoned that we could assess the impact of one isoform in the presence and absence of the other, on cell viability in a Bnip3-null background. As shown in FIG. 4F-G, Bnip3−/− MEF cells and Bnip3−/− MEFs expressing Bnip3Δex3 isoform were indistinguishable from each other with respect to cell viability. However, repletion of Bnip3FL into Bnip3−/− cells resulted in a significant increase in cell death. Importantly, cell death induced by Bnip3FL was rescued by coexpression of Bnip3Δex3. Collectively, our data strongly suggest that Bnip3Δex3 is an endogenous inhibitor that attenuates mitochondrial defects and cell death mediated by Bnip3FL during hypoxia.

To our knowledge, the data described herein provide the first demonstration that a member of the Bcl-2 gene family undergoes alternative splicing during hypoxia. Previously, we established that Bnip3 is crucial for provoking mitochondrial defects and cell death of ventricular myocytes during hypoxia (Regula et al., 2002, Circ. Res., 91:226-231, Shaw et al., 2008, Proc. Natl. Acad. Sci. U.S.A., 105:20734-20739, Shaw et al., 2006, Circ. Res., 99:1347-1354). In this report we provide new compelling evidence that alternative splicing of Bnip3 during hypoxia provides a molecular switch that determines whether Bnip3 triggers mitochondrial perturbations and cell death of cardiac myocytes. We specifically show that hypoxia not only drives Bnip3 transcription, but provides a molecular signal for alternative splicing of Bnip3 resulting in the exclusion of exon3. We determined that the Bnip3Δex3 mRNA resulting from the fusion of exon2 and exon4 introduces a frame shift mutation and stop codon that prematurely terminates transcription up-stream of exons and exon6, generating a truncated Bnip3 protein missing the putative BH3 domain and carboxyl terminal transmembrane domain required for mitochondrial targeting. Notably, in contrast to full length Bnip3, which provokes cell death, Bnip3Δex3 promotes cell survival. The underlying mechanisms that account for alternative splicing of Bnip3 pre-mRNA during hypoxia are unknown and are an active area of investigation; nevertheless, several salient and important findings arise from this work.

First, we found that Bnip3 gene transcription is not only activated in ventricular myocytes under relevant physiological conditions in vivo and in vitro, but hypoxia/ischemia provides a molecular signal that promotes alternative splicing of exon3 and synthesis of Bnip3Δex3. Second, and perhaps most compelling, were our findings demonstrating the ability of Bnip3Δex3 to interact and suppress the mitochondrial defects and cell death induced by Bnip3FL. Third, we show that cells defective for synthesizing Bnip3Δex3 displayed a marked increase in cell death during hypoxia.

Given that deregulated Bnip3 transcription would otherwise have catastrophic consequences in post-mitotic cells, implies that Bnip3 must be highly regulated and under tight transcriptional control. Indeed, we have previously demonstrated that the Bnip3 promoter is subject to strong basal repression, but is highly induced during hypoxia (Shaw et al., 2008, Proc. Natl. Acad. Sci. U.S.A., 105:20734-20739). However, despite the increase in Bnip3 transcription during hypoxia, certain cells can reportedly avert death in response to Bnip3 (Green et al., 1994, Important. Adv. Oncol., 1994:37-52, Kothari et al., 2003, Oncogene, 22:4734-4744). The underlying mechanism for this resistance is undetermined, but likely involves a mechanism that antagonizes the actions of Bnip3. This notion is supported by the fact that at no time did we detect full-length Bnip3 transcripts in the absence of truncated Bnip3Δex3. Furthermore, activating endogenous Bnip3 gene transcription under basal normoxic conditions was sufficient to increase the full-length Bnip3 isoform, but not the Bnip3 splice variant. This implies that alternative splicing of Bnip3 mRNA during hypoxia is a selective regulated process and may represent a novel cellular defense mechanism that safe-guards against indiscriminant mitochondrial damage and cell death that would otherwise occur by Bnip3 gene activation alone, if unopposed during hypoxia. This view is supported by the fact that we showed by not one, but by three, independent approaches that the extent of mitochondrial damage and cell death mediated by Bnip3FL was significantly greater in cells deficient for Bnip3Δex3. When taken together, our data strongly suggest that the principle function of Bnip3Δex3, at least operationally, is to limit or curtail mitochondrial damage and cell death induced by Bnip3FL during hypoxia. Though the mode by which Bnip3Δex3 suppresses cell death was not determined, the fact that mitochondrial associated Bnip3FL was reduced in the presence of Bnip3Δex3 strongly suggests it behaves as a dominant-negative inhibitor interfering with the mitochondrial targeting of Bnip3FL and/or its ability to provoke mitochondrial perturbations which if not curtailed would provoke cell death by apoptosis and/or necrosis pathways. This view is consistent with the loss of mitochondrial membrane potential and increased ROS production induced by Bnip3FL in cells deficient for Bnip3Δex3.

The closest homologue to Bnip3 is Nix/Bnip3L, which can reportedly undergo RNA splicing; however, unlike Bnip3, Nix is not induced in the heart during hypoxia or ischemia, but instead is transcriptionally activated by Gq-signaling during pathological cardiac hypertrophy (Yussman et al., 2002, Nat. Med., 8:725-730, Galvez et al., 2006, J. Biol. Chem. 281:1442-1448). Considering that the primary mode by which Bnip3-induces cell death is to disrupt mitochondrial function (Regula et al., 2002, Circ. Res., 91:226-231), it is possible that Bnip3FL may initially induce subtle mitochondrial changes still compatible with cell life, with more severely damaged or irreparable mitochondria removed from the cell by mitophagy. This would effectively postpone the induction of Bnip3-mediated apoptosis. This view is supported by a recent report documenting the dependency of Nix for efficient mitochondrial clearance in differentiating reticulocytes by ATG8/GABARAP (Dorn 2010, EMBO Rep. 11:45-51, Ding et al., 2010, J. Biol. Chem., 285:27879-27890, Kanki, 2010, Autophagy, 6:433-43533-36). However, this caveat must be interpreted with caution, since it remains to be tested whether signaling events involved in mitochondrial clearance in reticulocytes, as part of a normal developmental process, are equivalently operational in the myocardium (Dorn, 2010, J. Cardiovasc. TransL Res. 3:374-383). It is equally undetermined whether Bnip3FL plays any role in clearing damaged mitochondria during hypoxia, which was not addressed here and is beyond the scope of the present study. Nevertheless, the fact that Bnip3FL-induced mitochondrial perturbations were dramatically reduced in the presence of Bnip3Δex3 is consistent with this theory and a cytoprotective role for Bnip3Δex3.

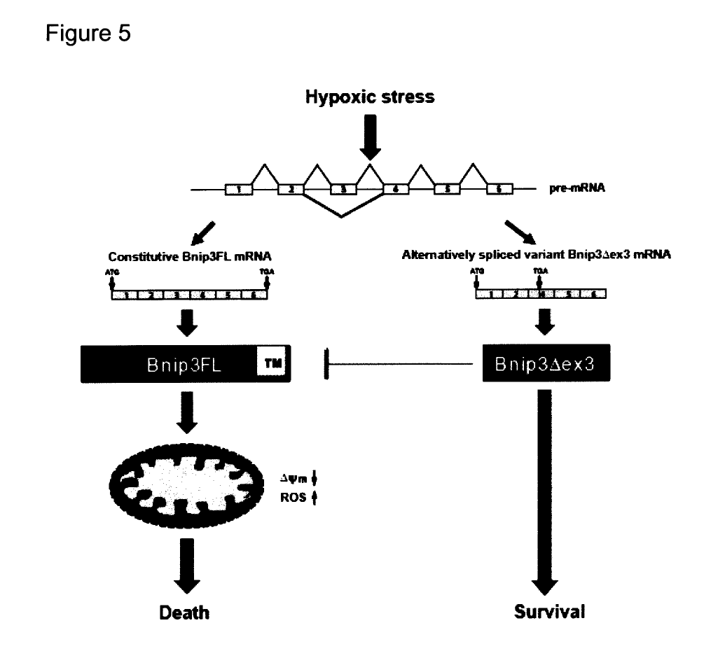

Therefore, based on the findings of the present study, we envision a model in which the synthesis of Bnip3Δex3 during hypoxia confers a survival role by opposing or dampening the mitochondrial defects induced by Bnip3FL; however, beyond a certain threshold Bnip3FL dominates and gives rise to irreversible mitochondrial injury and cell death (FIG. 5). This view is supported by the fact that ventricular myocytes rendered defective for Bnip3Δex3 were more sensitive to cell death induced by Bnip3FL and hypoxia. Based on our findings, we speculate the discordant and confounding reports on Bnip3FL's ability to provoke death in certain tumor cells may be explained in part by the unappreciated existence of the Bnip3Δex3 isoform, which we believe likely masks the ability of Bnip3FL to provoke cell death. This notion is supported by our findings demonstrating that knock-down of Bnip3Δex3 in pancreatic ductal carcinoma cells, which are resistant to hypoxic injury and which have elevated basal levels of the Bnip3Δex3 spliced variant, increased cell death in response to hypoxia.

Thus, our findings provide the first direct evidence for the existence of a novel survival mechanism that is obligatorily linked and mutually dependent upon hypoxia-induced alternative gene splicing of Bnip3. Hence, by curtailing the mitochondrial injury induced by Bnip3FL, Bnip3Δex3 variant may represent an adaptive mechanism to safe guard against excess Bnip3FL and cell death during hypoxic stress.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for modifying apoptosis, necrosis, autophagy, or the combination thereof, of a cell comprising:
expressing in a cell a polypeptide having Bnip3 antagonist activity, wherein (i) the amino acid sequence of the polypeptide and the amino acid sequence of SEQ ID NO:2 have at least 84% identity, or (ii) the amino acid sequence of the polypeptide and the amino acid sequence of SEQ ID NO:7 have at least 80% identity,
wherein apoptosis, necrosis, autophagy, or the combination thereof, is modified in the cell compared to a control cell.

2. The method of claim 1 wherein the expressing comprises introducing the polypeptide into the cell.

3. The method of claim 1 wherein the expressing comprises introducing into the cell a polynucleotide encoding the polypeptide.

4. The method of claim 3 wherein the polynucleotide comprises a vector.

5. The method of claim 1 wherein the cell is ex vivo.

6. The method of claim 1 wherein the cell is in vivo.

7. An isolated polypeptide having Bnip3 antagonist activity, wherein the polypeptide comprises an amino acid sequence having at least 80% sequence identity to SEQ ID NO: 2 or at least 80% sequence identity to SEQ ID NO:7.

8. The isolated polypeptide of claim 7 wherein the isolated polypeptide is a fusion polypeptide.

9. A composition comprising the isolated polypeptide of claim 7.

10. The composition of claim 9 wherein the composition further comprises a pharmaceutically acceptable carrier.

11. An isolated polypeptide comprising amino acids LRKMILKEGKKLKAS (SEQ ID NO:3).

12. The isolated polypeptide of claim 11 wherein the isolated polypeptide comprises between 1 and 3 conservative substitutions.

13. The isolated polypeptide of claim 11 wherein the isolated polypeptide consists of SEQ ID NO:3.

14. An isolated polypeptide comprising amino acids LRKIILREEEKLKVS (SEQ ID NO:8).

15. The isolated polypeptide of claim 14 wherein the isolated polypeptide consists of SEQ ID NO:8.

16. An isolated polynucleotide comprising: (a) a nucleotide sequence encoding a polypeptide having Bnip3 antagonist activity, wherein (i) the amino acid sequence of the polypeptide and the amino acid sequence of SEQ ID NO:2 have at least 80% identity, or (ii) the amino acid sequence of the polypeptide and the amino acid sequence of SEQ ID NO:7 have at least 80% identity, or (b) the full complement of the nucleotide sequence of (i) or (ii).

17. An isolated polynucleotide comprising: (a) a nucleotide sequence encoding a polypeptide having Bnip3 antagonist activity, wherein the polynucleotide has at least 80% identity to SEQ ID NO:1 or SEQ ID NO:6, or (b) the full complement of the nucleotide sequence of (a).

18. The isolated polynucleotide of claim 16 wherein the isolated polynucleotide comprises a heterologous polynucleotide.

19. The isolated polynucleotide of claim 18 wherein the heterologous polynucleotide comprises a regulatory sequence.

20. The isolated polynucleotide of claim 18 wherein the heterologous polynucleotide comprises a vector.

21. The isolated polynucleotide of claim 20 wherein the vector is a viral vector.

22. The isolated polynucleotide of claim 16 wherein the isolated polynucleotide is DNA.

23. A polynucleotide comprising between 18 and 30 nucleotides, wherein the nucleotide sequence of the isolated polynucleotide includes (a) nucleotides 197 and 198 of SEQ ID NO:1 and consecutive nucleotides selected from nucleotides 169 through 226 of SEQ ID NO:1, or the complement thereof, or (b) nucleotides selected from nucleotides 198-243 of SEQ ID NO:1, or the complement thereof.

24. The polynucleotide of claim 23 wherein the polynucleotide is RNA.

25. The polynucleotide of claim 24 wherein the RNA is double stranded.

26. An antibody that specifically binds a polypeptide comprising SEQ ID NO:2, wherein the antibody does not bind to a polypeptide comprising an amino acid sequence SEQ ID NO:4.

27. The antibody of claim 26 wherein the antibody is a monoclonal antibody.

28. An antibody that specifically binds a polypeptide comprising LRKMILKEGKKLKAS (SEQ ID NO:3).

29.-32. (canceled)

33. A method comprising:
administering to a subject in need thereof an effective amount of a composition comprising:
a) a polynucleotide comprising: a nucleotide sequence encoding a polypeptide having Bnip3 antagonist activity, wherein (i) the amino acid sequence of the polypeptide and the amino acid sequence of SEQ ID NO:2 have at least 80% identity, or (ii) the amino acid sequence of the polypeptide and the amino acid sequence of SEQ ID NO:7 have at least 80% identity, or
b) a polypeptide having Bnip3 antagonist activity, wherein (i) the amino acid sequence of the polypeptide and the amino acid sequence of SEQ ID NO:2 have at least 80% identity, or (ii) the amino acid sequence of the polypeptide and the amino acid sequence of SEQ ID NO:7 have at least 80% identity,
wherein apoptosis, necrosis, autophagy, or the combination thereof, is decreased in the subject.

34. The method of claims 33 wherein the polynucleotide is RNA.

35. The method of claim 34 wherein the RNA is double stranded.

36. The method of claim 33 wherein the polynucleotide comprises a vector.

37. The method of claim 36 wherein the vector is a viral vector.

38. The method of claim 33 wherein the administering comprises use of a catheter.

39. The method of claim 33 wherein the administering comprises delivery of the polynucleotide or the polypeptide to cardiac tissue or brain tissue.

40. The method of claim 33 wherein the subject has signs of or is at risk of a disease chosen from acute myocardial infarction, hypoxia, myocardial ischemia, myocardial infarction, stroke, or vascular disease.

41. The method of claim 40 wherein a sign of the disease is reduced.

42. A method comprising:
administering to a subject in need thereof an effective amount of a composition comprising:
a polynucleotide comprising between 18 and 30 nucleotides, wherein the nucleotide sequence of the isolated polynucleotide includes (a) nucleotides 197 and 198 of SEQ ID NO:1, or the complement thereof, or (b) nucleotides selected from nucleotides 198-243 of SEQ ID NO:1, or the complement thereof,
wherein cellular apoptosis, necrotic cell death, autophagy, or the combination thereof is increased in the subject.

43. The method of claim 40 wherein the polynucleotide is RNA.

44. The method of claim 43 wherein the RNA is double stranded.

45. The method of claim 42 wherein the polynucleotide comprises a vector.

46. The method of claim 45 wherein the vector is a viral vector.

47. The method of claim 40 wherein the administering comprises use of a catheter.

48. The method of claim 40 wherein the subject has signs or is at risk of cancer.

49. The method of claim 48 wherein the cancer is selected from pancreatic cancer, colon cancer, or breast cancer.

50. The method of claim 49 wherein a sign of the disease is reduced.

51. -75. (Canceled)

76. A method for identifying a compound that alters the amount or activity of a Bnip3Δex3 polypeptide in a cell, comprising:
exposing a cell to a compound; and
measuring the amount of Bnip3Δex3 polypeptide in the cell, the activity of Bnip3Δex3 polypeptide in the cell, or the combination thereof, wherein a change in the amount of Bnip3Dex3 polypeptide in the cell, the activity of Bnip3Δex3 polypeptide in the cell, or the combination thereof, compared to a control cell not exposed to the compound indicates the compound alters the amount or activity of Bnip3Δex3 polypeptide in the cell.

77. The method of claim 76 wherein the amount or activity of Bnip3Δex3 polypeptide in the cell is increased.

78. The method of claim 76 wherein the amount or activity of Bnip3Δex3 polypeptide in the cell is decreased.
